(19) United States
(12) Patent Application Publication
(10) Pub. No.: US 2024/0391876 A1
(43) Pub. Date: Nov. 28, 2024

(54) QUINACRINE AND DERIVATIVES THEREOF FOR TREATMENT OF VIRAL INFECTIONS

(71) Applicant: ARIZONA BOARD OF REGENTS ON BEHALF OF THE UNIVERSITY OF ARIZONA, Tucson, AZ (US)

(72) Inventor: Daekyu Sun, Tucson, AZ (US)

(21) Appl. No.: 18/684,808

(22) PCT Filed: Aug. 19, 2022
(86) PCT No.: PCT/US2022/075222

§ 371 Date: Feb. 19, 2024

Related U.S. Application Data
(60) Provisional application No. 63/234,903, filed on Aug. 19, 2021.

(51) Int. Cl. C07D 219/12; A61K 31/473; A61K 31/496; A61K 31/5377; A61P 31/14; C07D 219/10; C07D 401/12; C07D 413/12; C07D 417/12

(52) U.S. Cl. CPC C07D 219/12; A61K 31/473; A61K 31/496; A61K 31/5377; A61P 31/14; C07D 219/10; C07D 401/12; C07D 413/12; C07D 417/12

(57) ABSTRACT

Coronavirus Disease 2019 (COVID-19) is caused by severe acute respiratory syndrome coronavirus 2 (SARS-COV-2). The present invention reveals that quinacrine directly binds to G4 RNAs found in genomic RNA of the COVID-19 virus, showing antiviral activity against COVID-19 using in vitro cell culture systems. An object of the present invention is thus a new pharmaceutical composition of quinacrine for the treatment of COVID-19. Another object of the invention is a method for administering quinacrine by various routes specifically for treatment, including oral administration, sterile intravenous injection, and others.

### CROSS-REFERENCES TO RELATED APPLICATIONS

This application claims benefit of U.S. Provisional Application No. 63/234,903 filed Aug. 19, 2021, the specification of which is incorporated herein in their entirety by reference.

### FIELD OF THE INVENTION

The present invention relates to methods and compositions of preventing or treating the novel coronavirus SARS-COV-2, which causes COVID19, or other viral infections, more particularly, to a treatment method using quinacrine or a derivative of quinacrine that interacts with G-quadruplexes.

### BACKGROUND OF THE INVENTION

The novel severe acute respiratory syndrome coronavirus 2 (SARS-COV-2), which causes Coronavirus Disease 2019 (COVID-19), is a deadly zoonotic coronavirus with human-to-human transmission. Recent outbreaks of COVID-19 have caused much public concern due to its mortality rate and ease of transmission. Since the initial outbreak, there have been 580 million cases of COVID-19, with 6.4 deaths. However, COVID-19 is an ongoing pandemic and is expected to become a seasonal illness like many respiratory viruses (e.g., influenza). Thus, there remains a need for drugs to prevent or treat COVID-19. Here the present invention features quinacrine and derivatives thereof that interact with G-quadruplexes for the prevention or treatment of viral infections (e.g., COVID-19 and/or influenza).

### BRIEF SUMMARY OF THE INVENTION

It is an objective of the present invention to provide a method of administering a therapeutic amount of quinacrine for the prevention and treatment of a viral respiratory infection (such as COVID-19 and/or influenza), as specified in the independent claims. Embodiments of the invention are given in the dependent claims. Embodiments of the present invention can be freely combined with each other if they are not mutually exclusive.

Currently, the primary uses of quinacrine are as an antiprotozoal, antirheumatic, and intrapleural sclerosing agent. Presently, quinacrine's mechanism of action against protozoa is uncertain, but it is known to target the protozoan's cell membrane. Other known mechanisms of this drug are to act as a histamine N-methyltransferase inhibitor, inhibit NF-κB, and activate p53. Analysis done by the present invention determined that quinacrine is a compound that belongs to a G-quadruplex (G4)-interactive agent that can target RNA G4 structures. Quinacrine has also recently been revealed to directly bind to G4 RNAs found in genomic RNA of the COVID-19 virus, showing antiviral activity against COVID-19 using in vitro cell culture systems. An object of the present invention is thus a new pharmaceutical composition of quinacrine for the treatment of COVID-19. Another object of the present invention is a method for administering quinacrine by various routes specifically for treatment, including oral administration, sterile intravenous injection, and others.

In some embodiments, the present invention may feature a method of preventing or treating coronavirus disease 2019 (COVID-19) in a subject in need of such treatment. The method may comprise administering a therapeutic amount of quinacrine or a derivative thereof as described herein to the subject. Without wishing to limit the present invention to a particular theory or mechanism, the method may be capable of preventing or treating COVID-19 such that clinical improvement is observed.

Additionally, the present invention may feature a method of preventing or treating both coronavirus and influenza as well as other viral respiratory infections in a subject in need of such treatment. The method may comprise administering a therapeutic amount of quinacrine or a derivative thereof as described herein to the subject. Without wishing to limit the present invention to a particular theory or mechanism, the method may be capable of preventing or treating the virus/viral infection (e.g., coronavirus and/or influenza) such that clinical improvement is observed.

One of the unique and inventive technical features of the present invention is the use of quinacrine for the treatment and prevention of COVID-19, as well as other viral respiratory infections. Without wishing to limit the invention to any theory or mechanism, it is believed that the technical feature of the present invention advantageously provides for an effective method to suppress the proliferation of viruses (e.g., the SARS-COV-2 virus). The pharmacokinetics of quinacrine allows quinacrine to distribute into the lungs at concentrations that effectively suppress the virus. Additionally, quinacrine can be administered orally or intravenously.

None of the presently known prior references or work has the unique, inventive technical feature of the present invention. For example, the current treatment for COVID-19, remdesivir, is unlikely to achieve adequate clinical efficacy because remdesivir and its active metabolite are unlikely to be adequate in the lung to inhibit the SARS-COV-2 virus. Additionally, remdesivir can only be given intravenously.

Furthermore, the prior references teach away from the present invention. For example, hydroxychloroquine, an antimalarial drug, is in the same chemical family as quinacrine. However, hydroxychloroquine has been minimally successful in treating COVID-19 and was found to cause dangerous side effects, particularly to the heart. Despite being in the same chemical family as hydroxychloroquine, quinacrine has surprisingly been found to successfully treat COVID-19! Another surprising result of the present invention is that quinacrine can be used to treat different viral respiratory infections, such as COVID-19 and influenza, simultaneously.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, and 1C show the characteristics of G-quadruplexes (G4). FIG. 1A shows a general sequence formula for a potential G4 forming sequence. FIG. 1B shows a structure of a G-tetrad. FIG. 1C shows a cartoon representation of a parallel G4.

FIG. 2 shows eight G4 forming regions identified from the genome of the COVID-19 isolate (SARS-COV-2/61-TW/human/2020/NPL). The same sequences were identified in the genome of another COVID-19 isolate (2019-nCoV/USA-WA1/2020).

FIG. 3 shows a circular dichroism spectrum of 5 UM of SG1 and SG2 RNA G4 oligomers in 10 mM of Tris-HCl (pH 7.6) buffer containing 5 mM NaCl and 50 mM of KCl at 25° C.

FIG. 4 shows the chemical structure of the G4-interactive compound quinacrine.

FIG. 5 shows a Taq DNA polymerase assay to validate the stabilization of RET G-quadruplex by quinacrine. DNA polymerase stop assay was performed at increasing concentrations of each drug. Lanes M represents the di-deoxy sequencing G-reactions with the same template, which serves as the marker to locate the exact stop site. Bands P, S, and F represent the position of the free primer, stop products by G-quadruplex structures, and full-length products, respectively, on the gel.

FIG. 6 shows a CD titration spectrum for determining the melting curves of the RET G4 in the absence and presence of quinacrine (5 equivalent). In both graphs in FIG. 6, at the 260 wavelength lines in descending order are 20° C., 30° C., 40° C., 50° C., 60° C., 70° C., 80° C., and 90° C.

FIG. 7 shows an absorption spectrum of 40 M quinacrine (QC) in the presence of RNA G4 (SG1, SG2) at different concentrations in 10 mMTris-HCl buffer (pH 7.6) containing 5 mM NaCl and 50 mM of KCl.

FIG. 8 shows an antiviral effect of quinacrine evaluated by observing cytopathic effects (CPE). The cytoprotective effect of quinacrine was determined by the cell viability, which is measured by the uptake of neutral red dye at A540. At 120 h post-inoculation (p.i.), only in cells treated with 2 μg/mL quinacrine, CPE due to SARS-COV-2 was substantially reduced by more than 80% at the 30% cytotoxic concentration.

FIG. 9 shows thirteen G4 forming regions identified from the genome of the Influenza A virus.

FIG. 10 shows the antiviral effect of quinacrine against Influenza A/California/07/2009 (H1N1). The cytoprotective effect of quinacrine was determined by the cell viability, which is measured by the uptake of neutral red dye at A540. About 80-100% confluent Vero 76 cells were infected with Influenza A/California/07/2009 (H1N1) pdm09 stock to achieve the lowest possible multiplicity of infection (MOI) that would yield >80% cytopathic effect (CPE) within 3 days. Quinacrine was tested by incubating virus-infected cells at 37±2° C., 5% CO₂ for three days, and stained with neutral red dye for approximately 2 hours (±15 minutes to determine cytopathic effect).

FIG. 11 shows the general formula of the quinacrine derivatives of the present invention. Note: R₁: H, F, or Cl; R₂: H or CH₃; R₃: H, CH₃, or C₂H₅; and R₄: Aliphatic amines or nitrogen heterocycles-molecules containing nitrogen atom(s) along with carbon, oxygen, and/or sulfur.

FIG. 12 shows the synthesis of acridine scaffolds as precursors of quinacrine derivatives of the present invention. The target compounds 6-11 were first prepared as outlined in this figure according to the protocol described previously, in which N-arylanthranilic acid (group Ill compounds) was obtained from a reaction between each of group I compounds (1, 2, or 3) and group Il compounds (4 or 5) in combination followed by condensation of group III compounds with phosphorus oxychloride to obtain group IV compounds (6-chloro-acridines: 6, 7, 8, 9, 10, or 11).

FIG. 13 shows aromatic nucleophilic substitution with amino group substituents at position 6 of acridine scaffolds. The amino components used in the present invention were prepared by aromatic nucleophilic substitution on 6-chloroacridine derivatives with an excess of 1,3-diaminopropane, 3 chloropropylamine, 4-chlorobutan-2-amine, or 1-chloropentan-3-amine in established conditions well known in the art. Amine alkylation (amino-de-halogenation) is a type of organic reaction between an alkyl halide and an amine, which is also called nucleophilic aliphatic substitution (of the halide), and the reaction product is a higher substituted amine. The aliphatic amination of aryl halides was done by using the most widely employed method for such carbon-nitrogen (C—N) bond-forming processes, in which copper, palladium, and nickel metal salts were used as catalysts. The Ullman coupling reaction of an aryl halide with an amine to form a C—N bond in the presence of a copper catalyst was used.

FIG. 14 shows the synthetic strategy for aliphatic amination of acridine halides. Each of group IV compounds (compound 6, 7, 8, 9, 10, or 11) was reacted with compounds 12, 13, 14, or 15 in combination for the aliphatic amination of acridine 6-chloride to produce organic compounds containing the N-acridine moiety in the present application as previously described. N-Alkylation of amines and nitrogen-containing heterocyclic compounds with alkyl halides (group 13, 14, or 13 compounds). Aliphatic amines were alkylated at N-1 using alkyl halides (group 13, 14, or 13 compounds) to provide N-alkyl-heterocycles. The nucleophilic substitution of alkyl halides with aliphatic amines is a traditional organic reaction. It is a relatively simple, well-established reaction and still a primary method for the alkylation of amines with aliphatic compounds.

FIG. 15 shows an alkylation of aliphatic amines with group 13 compounds.

FIG. 16 shows an alkylation of aliphatic amines with group 14 compounds.

FIG. 17 shows an alkylation of aliphatic amines with group 15 compounds.

FIG. 18 shows an example of the alkylation of morpholine with alkyl halides to obtain compounds described in this invention. Morpholine was alkylated with each alkyl halide (group 13, 14, and 15 compounds) using the usual synthetic method to obtain claimed compounds in the present invention. Alkylation of Nitrogen(s) containing heterocycles at N-1 with alkyl halides (group 13, 14, or 13 compounds). Nitrogen(s) containing heterocycles were alkylated at N-1 using alkyl halides (group 13, 14, or 13 compounds) to provide N-alkyl-heterocycles. The nucleophilic substitution of alkyl halides with heteroaromatic amines is a traditional organic reaction. It is a relatively simple, well-established reaction and still a primary method for the alkylation of amines with aliphatic compounds. The alkylation of the nitrogen atom of thiazoles and oxazole by group 13, 14, or 13 compounds was also done to give the corresponding alkylated products at the nitrogen atom.

FIG. 19 shows examples of alkylation of different heterocycles (X) at N-1 with alkyl halides (group 13, 14, or 13 compounds) to obtain the compounds claimed in the present invention.

FIGS. 20A, 20B, 20C, and 20D show analysis of selected compounds described in this invention using SwissADME to examine the compound's probability of being an oral drug and to evaluate the pharmacokinetics, drug-likeness, and medicinal chemistry friendliness of small molecules.

### DETAILED DESCRIPTION OF THE INVENTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely.” Furthermore, variation of the word “comprising”, such as “comprise” and “comprises,” have correspondingly the same meanings. In one respect, the technology described herein related to the herein described compositions, methods, and respective component(s) thereof, as essential to the invention, yet open to the inclusion of unspecified elements, essential or not (“comprising”).

As used herein, “coronavirus” may refer to a group of related viruses such as but not limited to severe acute respiratory syndrome (SARS), middle east respiratory syndrome (MERS), and severe acute respiratory syndrome coronavirus 2 (SARS-COV-2). All the coronaviruses cause respiratory tract infection that ranges from mild to lethal in mammals. As used herein, “Coronavirus Disease 19 (COVID-19)” is a novel coronavirus that causes an upper respiratory infection in patients. COVID-19 is caused by the severe acute respiratory syndrome coronavirus 2 (SARS-COV2) virus. It is spread primarily through droplets generated when an infected person coughs or sneezes or through droplets of saliva or discharge from the nose.

As used herein, “influenza” is an infectious disease caused by an influenza virus. It is spread primarily through droplets generated when an infected person coughs or sneezes, or through droplets of saliva or discharge from the nose. In some embodiments, an influenza virus may include but is not limited to Influenza A virus, Influenza B virus, Influenza C virus, or Influenza D virus.

As used herein, “patient” or “subject” to be treated includes humans and or non-human animals, including mammals, of any age and sex. Mammals include primates, such as humans, chimpanzees, gorillas and monkeys, and domesticated animals.

As used herein, “G-quadruplex” or “G4” may be used interchangeably. In some embodiments, G4 may refer to secondary structures that are formed in nucleic acids by sequences that are rich in guanine. They are helical in shape and contain guanine tetrads that can form from one, two, or four strands.

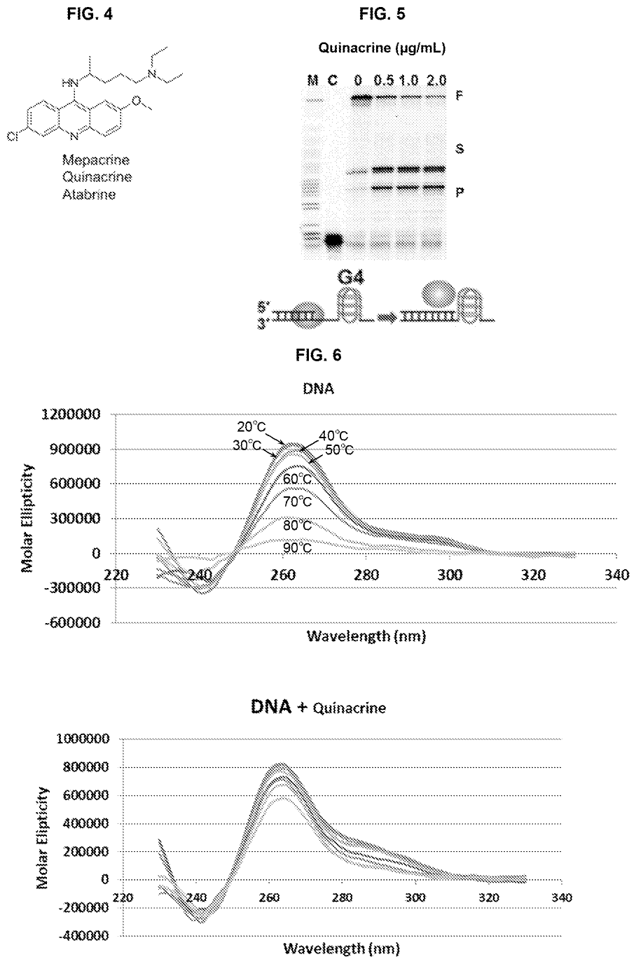

The terms “quinacrine,” “mepacrine,” or “atabrine” may be used interchangeably. Referring to FIG. 4, in some embodiments, quinacrine is a G4-interactive agent and can target RNA G4 structures.

As used herein, “clinical improvement” may refer to a noticeable reduction in symptoms of a disorder or disease or cessation thereof.

The terms “administering” and “administration” refer to methods of providing pharmaceutical compositions to a subject. Such methods are well known to those skilled in the art and include, but are not limited to, administering the compositions orally, parenterally (e.g., intravenously or subcutaneously), by intramuscular or intraperitoneal injection, intrathecally, transdermally, extracorporeally, topically or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves the use of a slow release or sustained release system such that a constant dosage is maintained. See, for example, U.S. Pat. No. 3,610,795, which is incorporated by reference herein.

A “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary and can be administered in one or more dose administrations daily, for one or several days. Guidance can be found in the literature for appropriate dosages for given classes of pharmaceutical products.

The compositions described herein can be administered in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as known to one of skill in the art.

Pharmaceutical carriers are known to those skilled in the art. These most typically would be standard carriers for the administration of drugs to humans, including solutions such as sterile water, saline, and buffered solutions at physiological pH. Typically, an appropriate amount of pharmaceutically acceptable salt is used in the formulation to render the formulation isotonic. Examples of the pharmaceutically acceptable carrier include, but are not limited to, saline, Ringer's solution, and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents, and the like, in addition to the compounds disclosed herein. Pharmaceutical formulations can also include one or more additional active ingredients such as antimicrobial agents, anti-inflammatory agents, anesthetics, and the like.

The pharmaceutical formulation can be administered in a number of ways depending on whether local or systemic treatment is desired and on the area to be treated. A preferred mode of administration of the composition is oral. Other modes of administration may be parenteral, for example, by intravenous drip, subcutaneous, intraperitoneal, or intramuscular injection. The disclosed compounds can be administered orally, intravenously, intraperitoneally, intramuscularly, subcutaneously, or transdermally.

Pharmaceutical compositions for oral administration include, but are not limited to, powders or granules, suspensions or solutions in water or non-aqueous media, capsules, sachets, or tablets. Thickeners, flavorings, diluents, emulsifiers, dispersing aids, or binders may be desirable. Quinacrine can be administered to a subject orally in a dosage taken once daily or in divided doses. A person of skill monitoring a subject's clinical response can adjust the frequency of administration of the medication according to methods known in the art.

Preparations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose, and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, inert gases, and the like.

Before the present compounds, compositions, and/or methods are disclosed and described; it is to be understood that this invention is not limited to specific synthetic methods or to specific compositions, as such may, of course, vary. It is also to be understood that the terminology used herein is for the purpose of describing particular embodiments only and is not intended to be limiting.

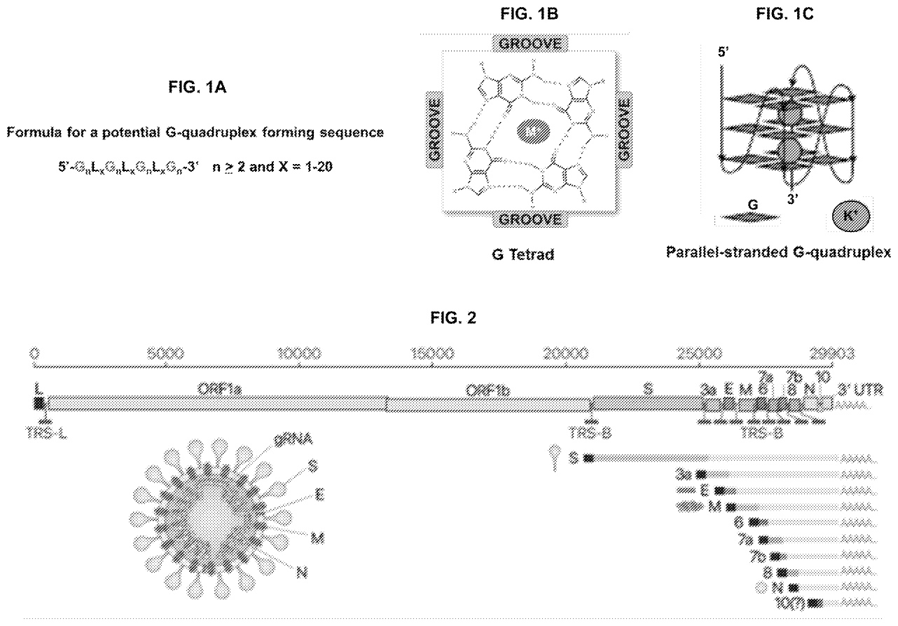

G-quadruplex (G4) structures are one of many categories of non-B-form nucleic acid structures that can form within specific repetitive guanine (G)-rich DNA or RNA both in vitro and in vivo (FIGS. 1A-1C). G4s are extremely stable secondary structures formed by a G-quartet where four guanine bases (G-tetrad) interact with each other via H-bonding and are further stabilized by a central monovalent cation, such as K⁺ or Na⁺ (FIG. 1B). Stable DNA G4 structures are found in sub-telomeres and gene bodies. A G4 antibody was used to map the location of such structures in human genomic DNA using immunoprecipitation followed by deep sequencing of the selected DNA fragments. The results of these studies suggest that DNA G4 in vivo may play an important role in several biological events, including transcription, replication, and recombination. The nuclear helicases, WRN (Werner syndrome ATP-dependent helicase), and Bloom syndrome protein are involved in resolving DNA G-quadruplexes in the genome; these structures are believed to be potentially damaging to a cell. However, no cytoplasmic cellular enzymes have been identified to resolve RNA G4s. Furthermore, RNA G4s are more prevalent in vivo, as RNA largely exists in a single-stranded conformation rather than the double-stranded conformation common to DNA. These features of RNA G4s make them more susceptible to small molecule G4 ligands than DNA G4s.

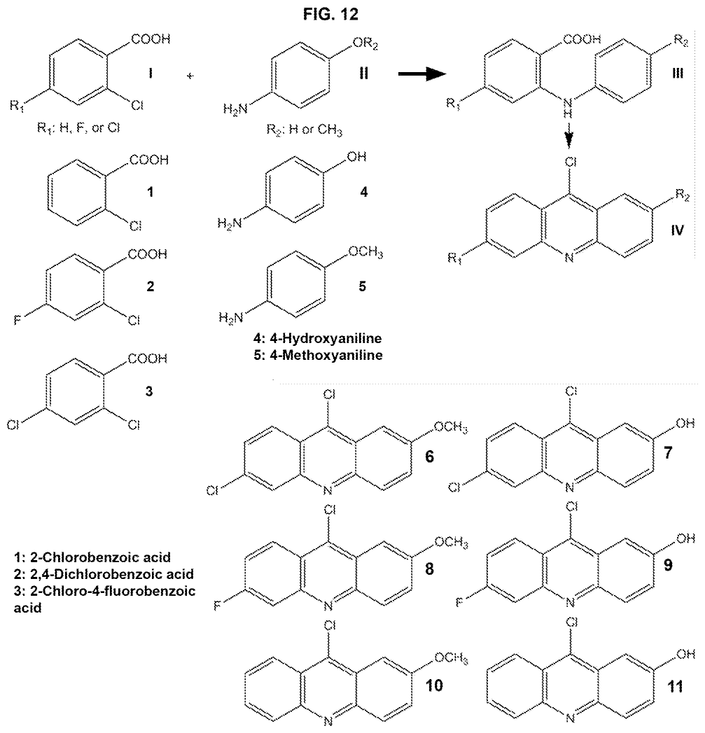

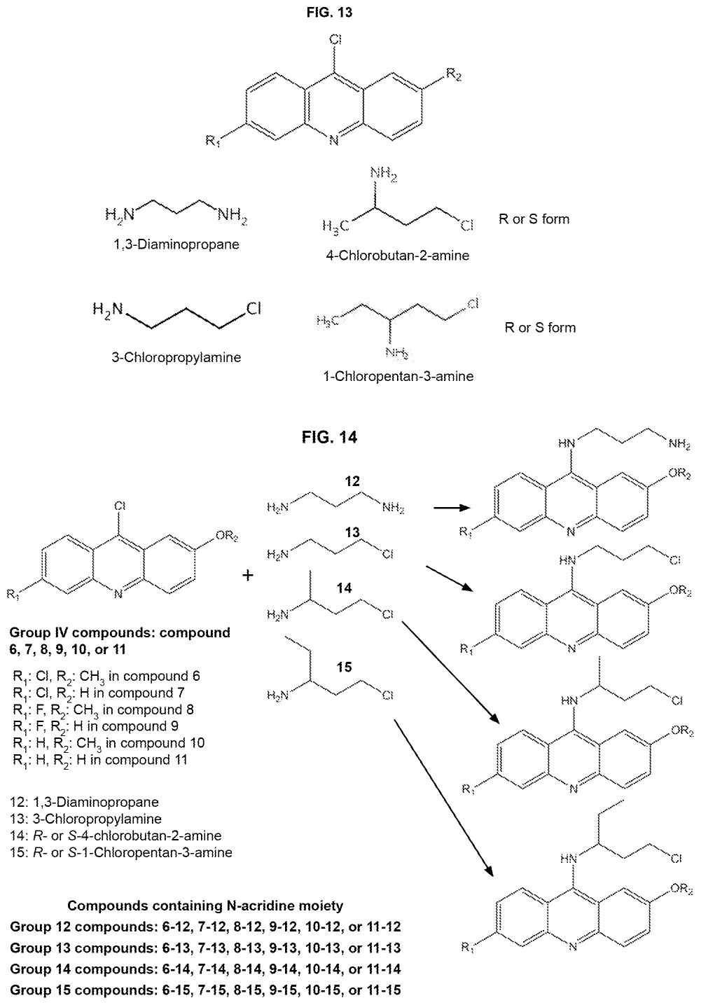

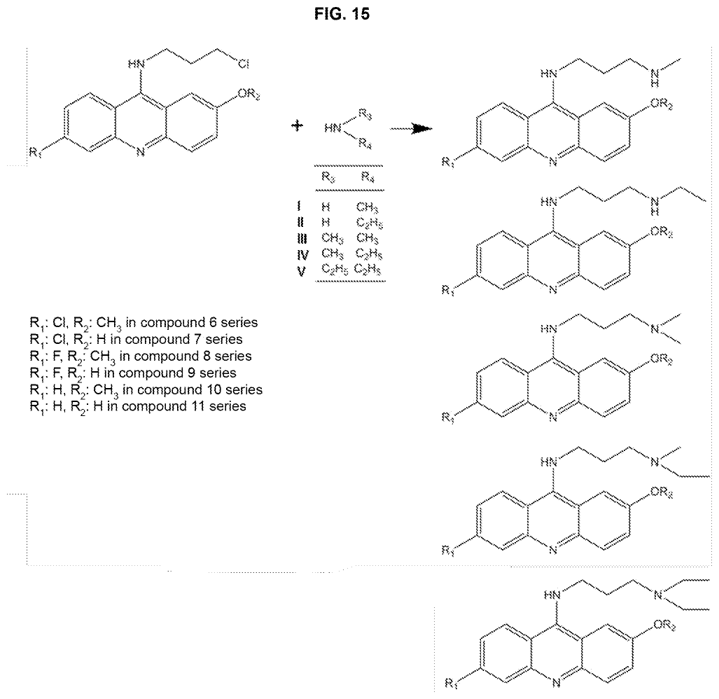

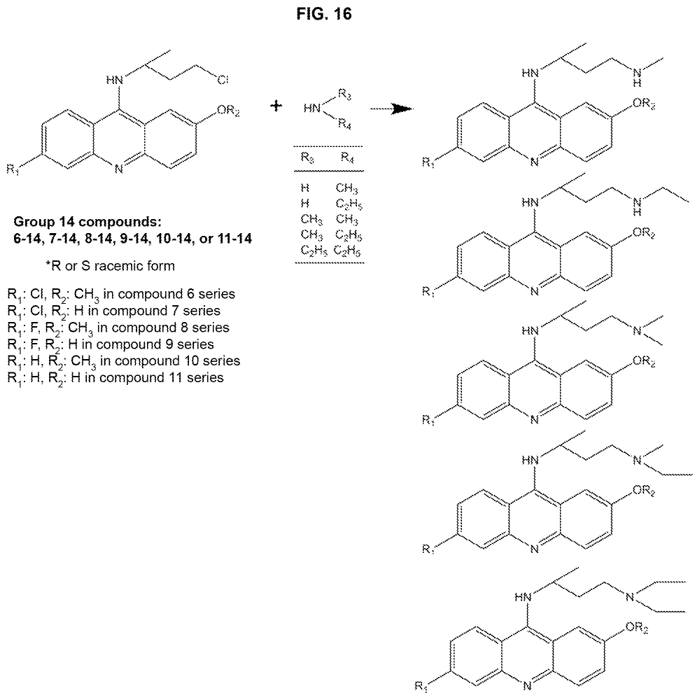

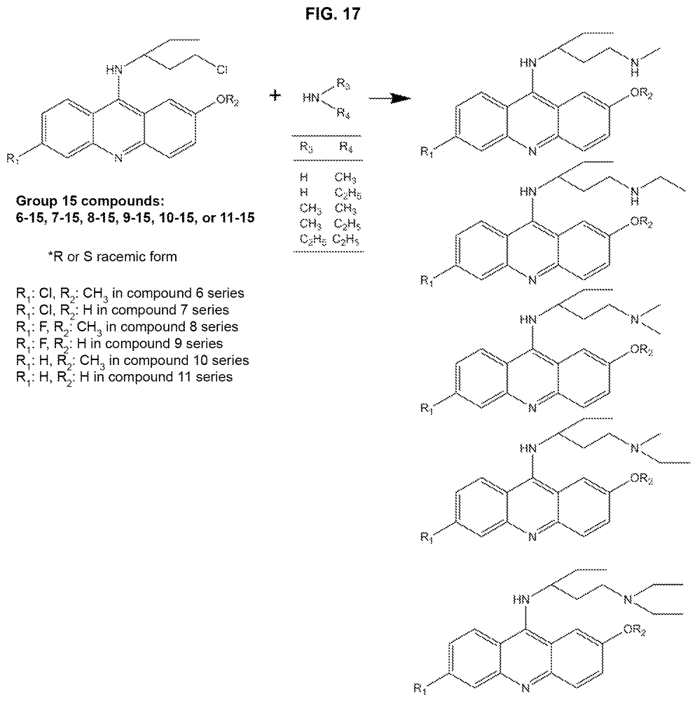

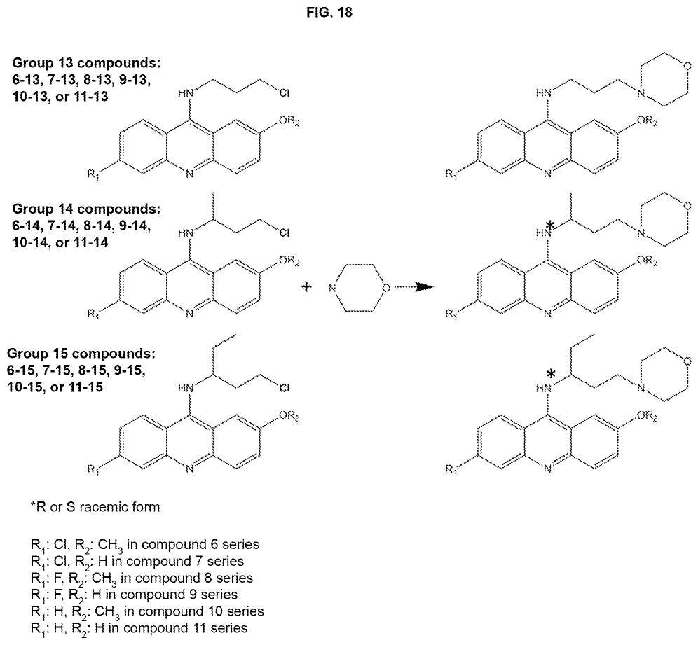

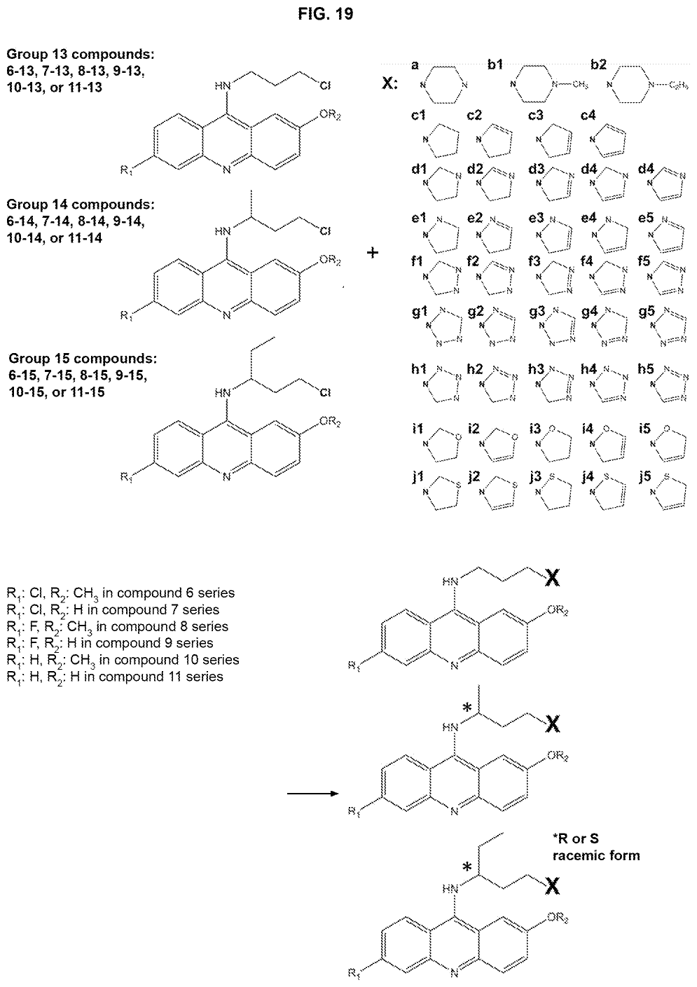

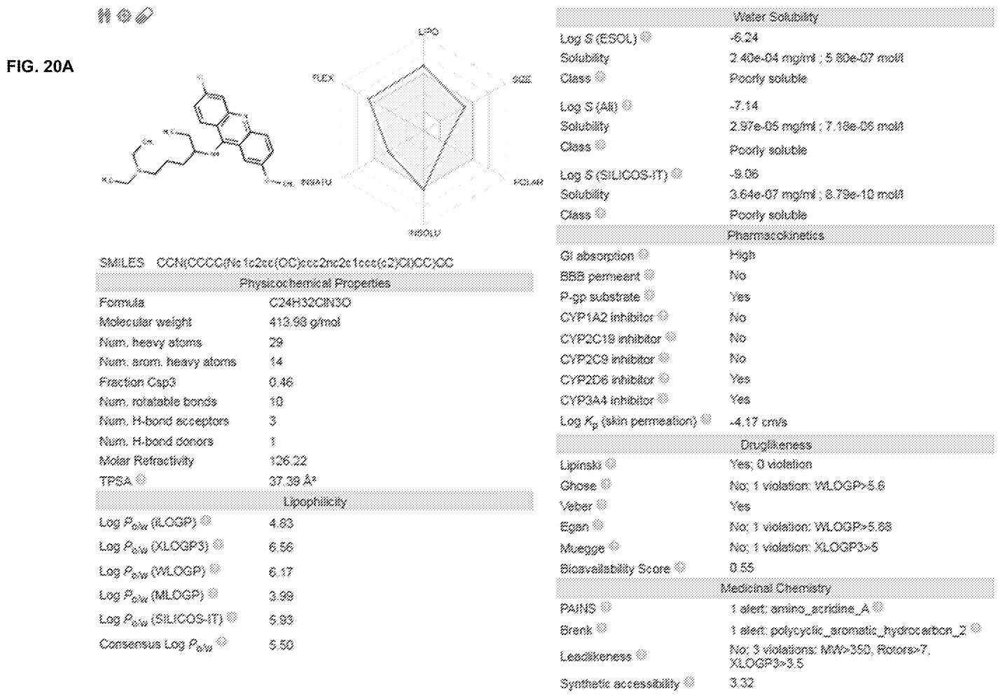

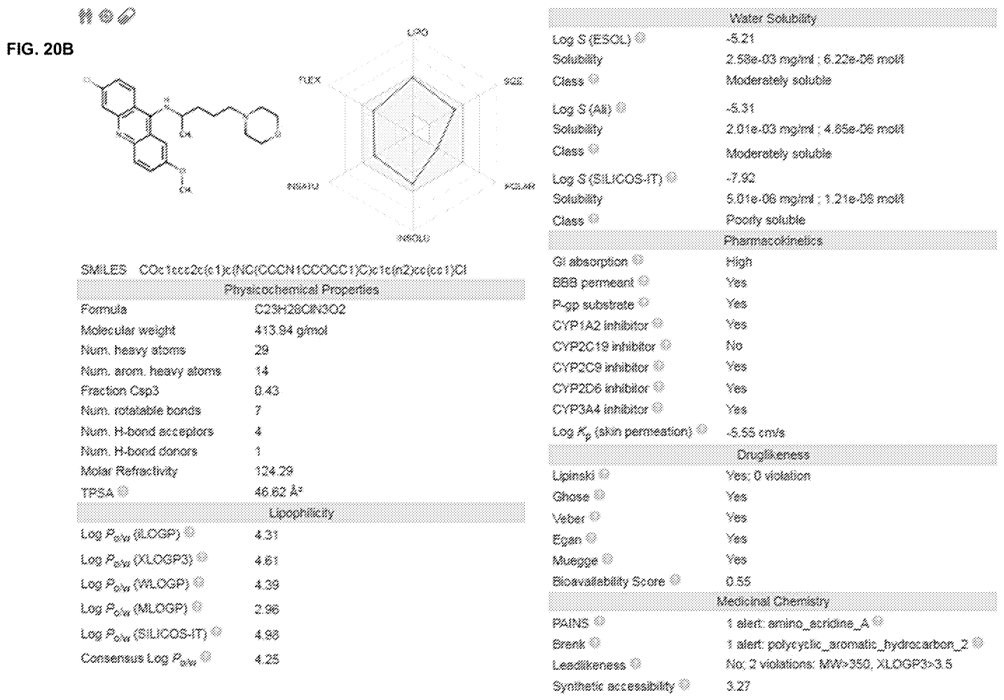

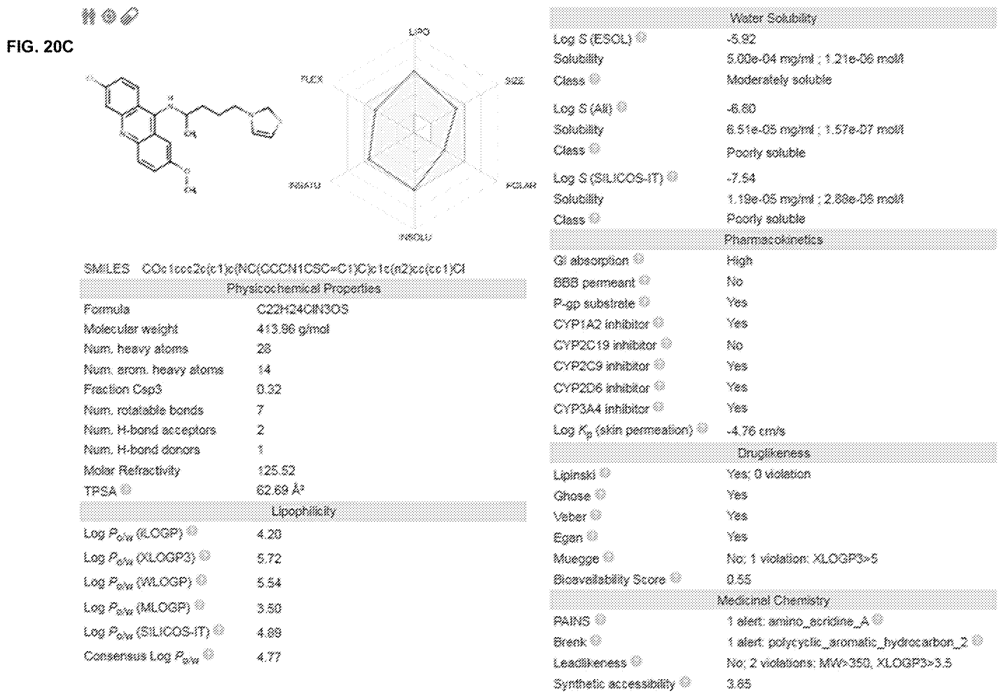

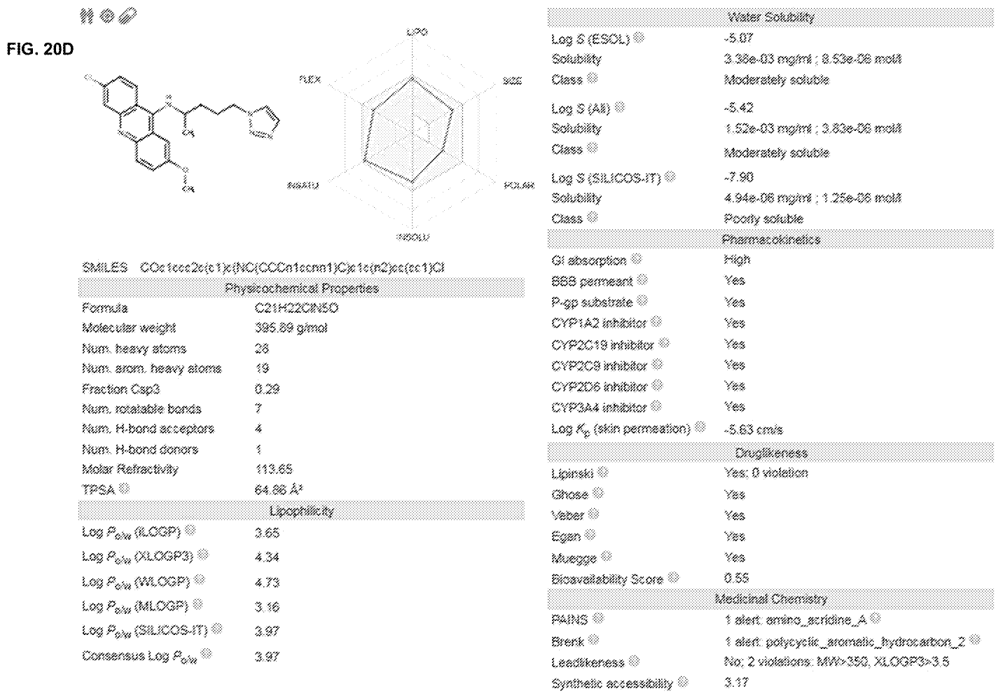

Referring now to FIGS. 1A-20D, the present invention, may feature methods of preventing or treating viral infections (e.g., coronavirus SARS-COV-2, which causes COVID19, or influenza), more particularly, a treatment method using quinacrine or a derivative of quinacrine that interacts with G-quadruplexes.

The present invention may feature a method of preventing and/or treating a viral respiratory infection in a subject in need of such treatment. The method may comprise administering to the subject a therapeutic amount of quinacrine. In some embodiments, the method is capable of preventing and/or treating a viral respiratory infection such that clinical improvement is observed.

As used herein, a “viral respiratory infection” is an infection that affects the lungs and airways. In some embodiments, a viral respiratory infection is caused by a virus. In other embodiments, a viral respiratory infection is caused by an RNA virus. In some embodiments, the RNA virus is a positive-strand RNA virus. In some embodiments, the RNA virus is a negative-strand RNA virus.

In some embodiments, the viral respiratory infection may be caused by one or more viruses. In some embodiments, the viral respiratory infection is caused by severe acute respiratory syndrome-associated coronavirus (SARS-COV; e.g., SARS-COV1 or SARS-COV2) virus, middle east respiratory syndrome (MERS) virus, an influenza virus, respiratory syncytial virus, or a combination thereof. Without wishing to limit the present invention to any theories or mechanisms, it is believed that the respiratory infections caused by the aforementioned coronaviruses range from the common cold to more severe diseases such as severe acute respiratory syndrome (SARS), Middle East respiratory syndrome (MERS), and COVID-19, which can be safely and effectively treated with compositions described herein.

In some embodiments, the present invention may also feature a method of preventing or treating coronavirus disease 2019 (COVID-19) in a patient in need of such treatment. The method may comprise administering a therapeutic amount of quinacrine or a derivative thereof as described herein to the patient. Without wishing to be bound to a particular theory or mechanism, it is believed that the method may be capable of preventing or treating COVID-19 such that clinical improvement is observed.

According to one embodiment, the present invention may feature a method of simultaneously preventing and/or treating coronavirus disease 2019 (COVID-19) in a patient in need of such treatment. The method may comprise administering a therapeutic amount of quinacrine or a derivative of quinacrine as described herein to the patient. In some embodiments, the method is capable of preventing or treating COVID-19 such that clinical improvement is observed.

The present invention may further feature a method of preventing and/or treating both coronavirus and influenza in a subject in need of such treatment. The method may comprise administering a therapeutic amount of quinacrine or a derivative thereof to the patient. In some embodiments, the method is capable of preventing and/or treating coronavirus and influenza such that clinical improvement is observed

In other embodiments, the present invention features methods and compositions for preventing or treating a viral respiratory infection (e.g., coronavirus disease 2019 (COVID-19) or influenza) in a patient in need of such treatment. The compositions may comprise quinacrine or a derivative thereof. Additionally, the compositions described herein may comprise a high oral bioavailability and/or antiviral activity (e.g., anti-COVID-19 activity). In some embodiments, the derivatives and compositions described herein are particularly well targeted for treating COVID-19 infections. These derivatives can be easily prepared from readily available starting materials utilizing routine synthetic procedures.

As used herein, a “high oral bioavailability” may refer to a composition having greater than 10% bioavailability in rats or an oral bioavailability value of 0.55 based on Lipinski's rules with passive intestinal absorption as a function of lipophilicity.

In some embodiments, quinacrine, or a derivative thereof, may be used in combination with other drugs. In some embodiments, quinacrine or a derivative thereof, as described herein, may be used in combination with other drugs that have different modes of action. Non-limiting examples may include but are not limited to remdesivir, hydroxychloroquine, leronlimab, ivermectin, nirmatrelvir, ritonavir, oseltamivir phosphate, zanamivir, peramivir, baloxavir marboxil, or a combination thereof. In some embodiments, quinacrine may be used in combination with other antiviral agents, immunotherapies, and vaccines.

Without wishing to limit the present invention to any theories or mechanisms, it is believed that combining compositions described herein with other drugs (e.g., conventional therapies and/or traditional therapies) with different modes of action may be more effective than the drug alone in methods of treatment. For example, it is believed that a combination of remdesivir IV and quinacrine regimen could be a potentially more effective antiviral therapy against COVID-19. Additionally, the use of quinacrine in combination with other drugs could be a more effective antiviral therapy against COVID-19.

In some embodiments, quinacrine or a derivative thereof as described herein for use may be administered once daily or twice daily. In another embodiment, quinacrine or a derivative thereof as described herein may be administered at least once to four times daily. In some embodiments, quinacrine or a derivative thereof as described herein may be administered at least once daily, at least once every other day, or at least once weekly. In another embodiment, quinacrine or a derivative thereof as described herein may be administered continuously by an intravenous drip. In other embodiments, quinacrine or a derivative thereof as described herein is administered at a daily dose ranging from about 2 mg/kg body weight to 10 mg/kg body weight. Further still, quinacrine or a derivative thereof as described herein may be administered intravenously or orally. In preferred embodiments, quinacrine or a derivative thereof as described herein for use in the treatment resulted in clinical improvement of COVID-19 and/or influenza.

In another embodiment, quinacrine or a derivative thereof is administered in a dosage of about 50 mg to 1000 mg. For example, the dosage may range from about 50 mg to 1000 mg, with a preferred range of about 100 mg to 800 mg for administration intravenously or a preferred range of 100 mg to 800 mg for administration orally. Quinacrine may be administered once daily or twice daily, or three or four times daily; or quinacrine may be administered once to four times daily; or quinacrine may be administered at least once daily, at least once every other day, or at least once weekly; or quinacrine may be administered continuously. In further embodiments, the composition may be administered orally or intravenously.

In any of the aforementioned embodiments of the present invention, quinacrine or a derivative of quinacrine may be administered in a dosage of about 0.5 mg/kg body weight to 20 mg/kg body weight. For example, the dosage may range from about 0.5 mg/kg body weight to 1 mg/kg body weight, or about 1 mg/kg body weight to 2 mg/kg body weight, or about 2 mg/kg body weight to 3 mg/kg body weight, or about 3 mg/kg body weight to 4 mg/kg body weight, or about 4 mg/kg body weight to 5 mg/kg body weight, or about 5 mg/kg body weight to 6 mg/kg body weight, or about 6 mg/kg body weight to 7 mg/kg body weight, or about 7 mg/kg body weight to 8 mg/kg body weight, or about 8 mg/kg body weight to 9 mg/kg body weight, or about 9 mg/kg body weight to 10 mg/kg body weight, or about 10 mg/kg body weight to 12 mg/kg body weight, or about 12 mg/kg to 14 mg/kg body weight, or about 14 mg/kg body weight to 16 mg/kg body weight or about 16 mg/kg body weight to 18 mg/kg body weight, or about 18 mg/kg body weight to 20 mg/kg body weight.

In any of the aforementioned embodiments of the present invention, quinacrine or a derivative of quinacrine may be administered in a dosage of about 50 mg to 1000 mg per day. For example, the dosage may range from about 50 mg/day to 100 mg/day, or about 100 mg/day to 150 mg/day, or about 150 mg/day to 200 mg/day, or about 200 mg/day to 250 mg/day, or about 250 mg/day to 300 mg/day, or about 300 mg/day to 350 mg/day, or about 350 mg/day to 400 mg/day, or about 400 mg/day to 450 mg/day, or about 450 mg/day to 500 mg/day, or about 500 mg/day to 550 mg/day, or about 550 mg/day to 600 mg/day, or about 600 mg/day to 650 mg/day, or about 700 mg/day to 750 mg/day, or about 750 mg/day to 800 mg/day, or about 800 mg/day to 850 mg/day, or about 850 mg/day to 900 mg/day, or about 900 mg/day to 950 mg/day, or about 950 mg/day to 1000 mg/day.

In one embodiment, the subject may be a mammal, such as a human. In another embodiment, the composition as described herein is administered in a dosage of about 50 mg to 1000 mg. For example, the dosage may range from 50 mg to 1000 mg with a preferred range of about 100 mg to 800 mg The composition as described herein may be administered once daily or twice daily or three or four times daily; or the composition as described herein may be administered at least once daily, at least once every other day, or at least once weekly or once monthly. In further embodiments, the composition as described herein may be administered orally or intravenously.

In some embodiments, the composition for use may be administered once daily or twice daily. In another embodiment, the composition may be administered at least once daily, at least once every other day, at least once weekly, or once monthly. Further still, the composition may be administered intravenously or orally. In preferred embodiments, the composition for use in the treatment resulted in clinical improvement of COVID-19. For example, clinical improvement may be observed in about 1 to 7 days or about 7 to 14 days or about 14-21 days, or about 21-28 days.

In some embodiments, the present invention may feature a composition comprising a derivative of quinacrine:

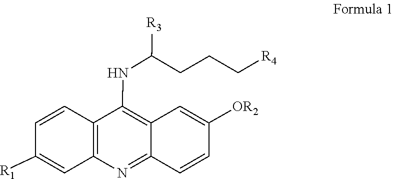

In some embodiments, R1 comprises an H, F, or Cl group. In some embodiments, R2 comprises an H or an alkyl group. In some embodiments, R3 comprises an H or an alkyl group. In some embodiments, R4 comprises an aliphatic amine or a nitrogen heterocyclic group. In other embodiments, the aliphatic amine or the nitrogen heterocyclic group comprises nitrogen, carbon, oxygen, and/or sulfur molecules.

In some embodiments, compositions (i.e., quinacrine derivatives) described herein may include but are not limited to:

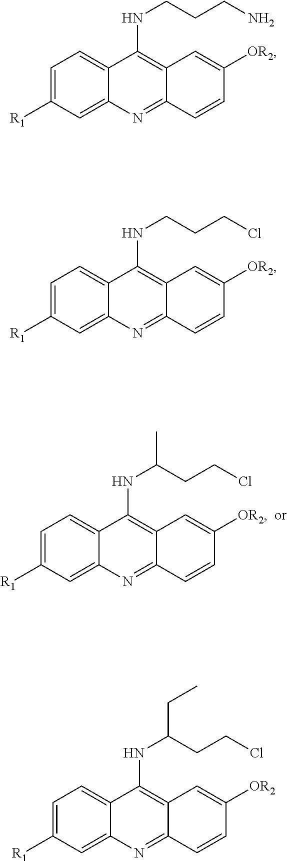

In other embodiments, compositions (i.e., quinacrine derivatives) described herein may include but are not limited to:

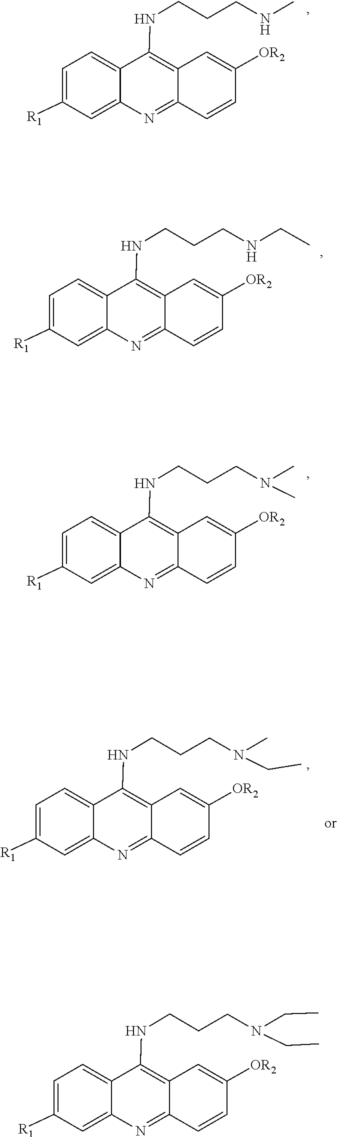

In some embodiments, compositions (i.e., quinacrine derivatives) described herein may include but are not limited to:

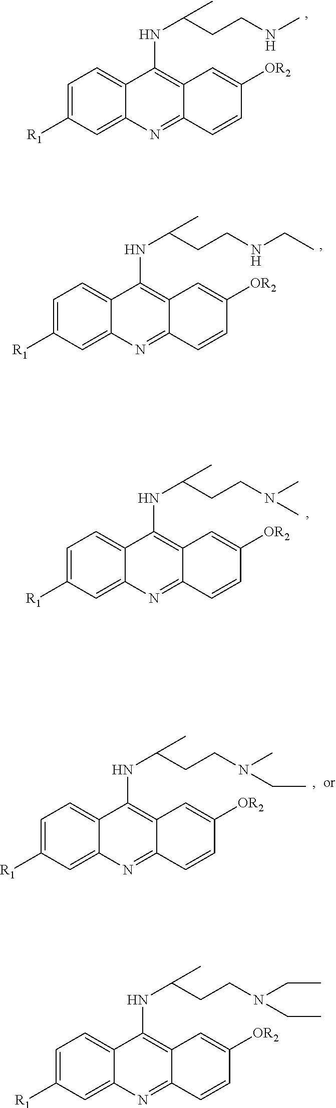

In other embodiments, compositions (i.e., quinacrine derivatives) described herein may include but are not limited to:

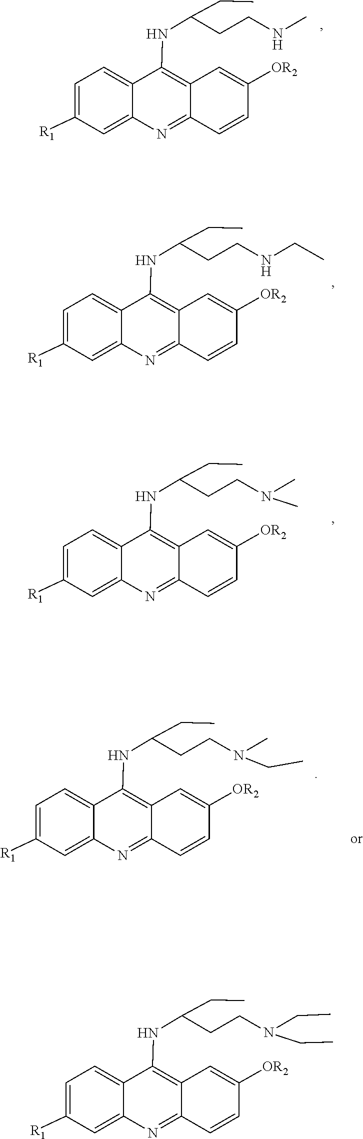

In some embodiments, compositions (i.e., quinacrine derivatives) described herein may include, but are not limited to:

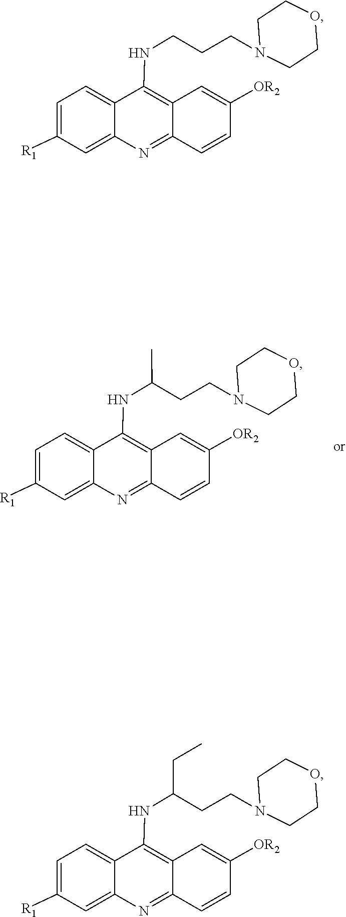

In accordance with the aforementioned embodiments, R1 comprises an H, F, or Cl group. In other embodiments, R2 comprises H or an alkyl group.

In other embodiments, compositions (I.e., quinacrine derivatives) described herein may include but are not limited to:

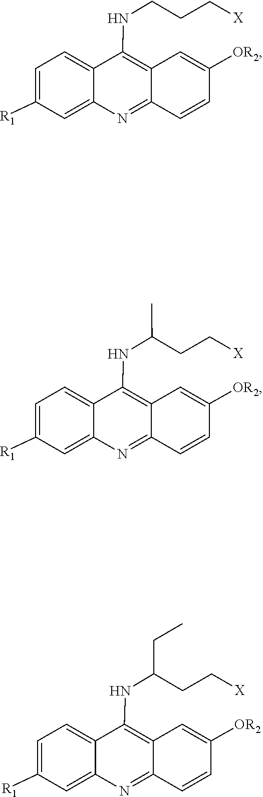

In some embodiments, R1 comprises an H, F, or Cl group. In other embodiments, R2 comprises H or an alkyl group. In some embodiments, X comprises a heterocyclic compound. In other embodiments, the heterocyclic compound includes but is not limited to:

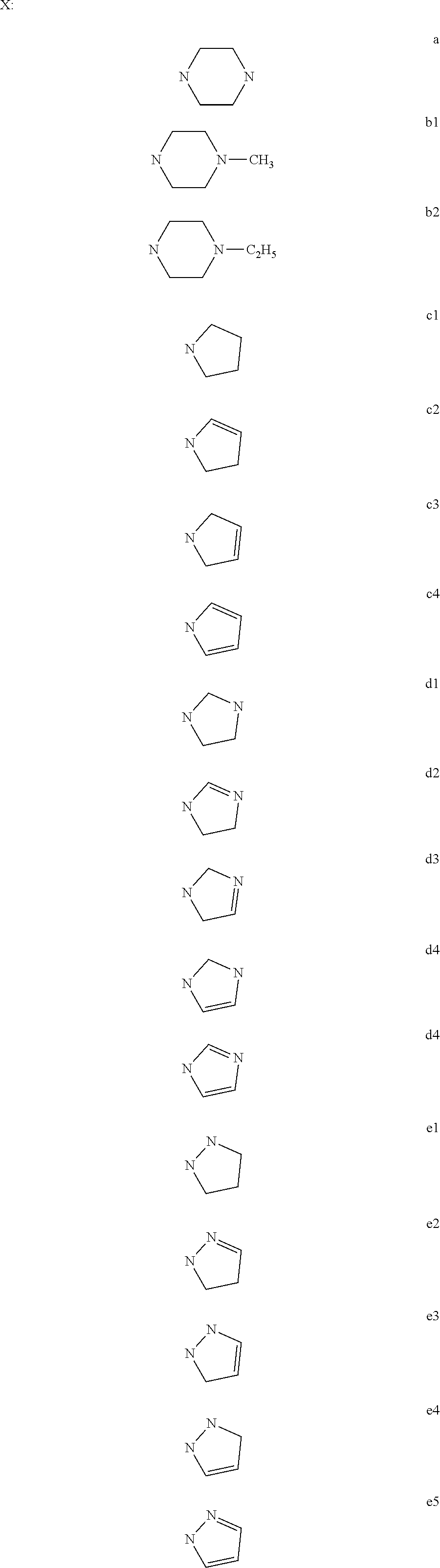

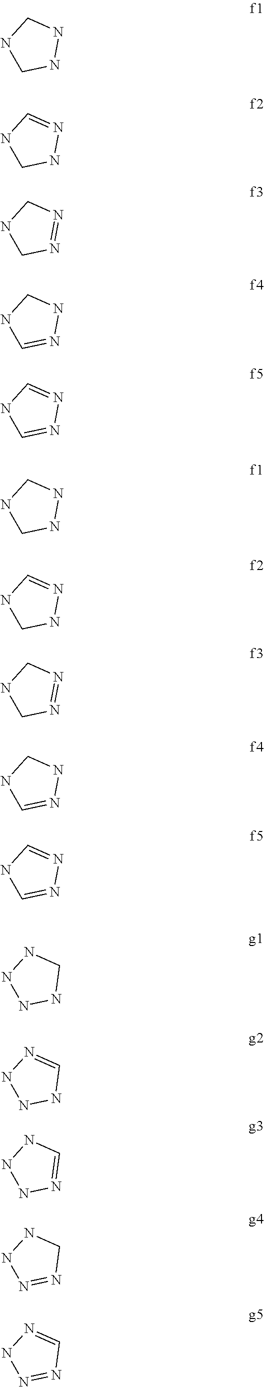

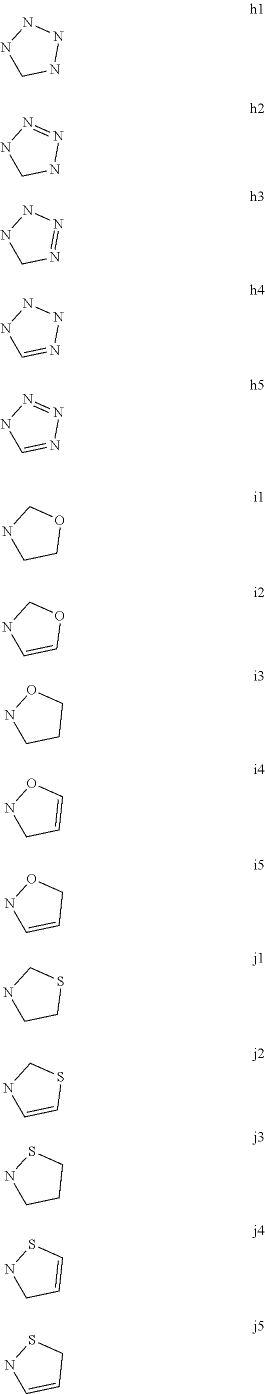

In some embodiments, the present invention features a method of preventing or treating coronavirus disease 2019 (COVID-19) in a subject in need of such treatment. In other embodiments, the present invention may also feature a method of simultaneously treating both coronavirus disease 2019 (COVID-19) and influenza in a subject in need of such treatment. In further embodiments, the present invention features a method of treating a viral respiratory infection in a subject in need of such treatment. In some embodiments, the method comprises administering to the subject a therapeutic amount of a composition as described herein (e.g., quinacrine derivatives). In some embodiments, the compositions described herein (e.g., quinacrine derivatives) are used in combination with other drugs.

### EXAMPLE

The following is a non-limiting example of the present invention. It is to be understood that said example is not intended to limit the present invention in any way. Equivalents or substitutes are within the scope of the present invention.

### Example 1.1 RNA G4 Structures of COVID-19 Genomic RNA

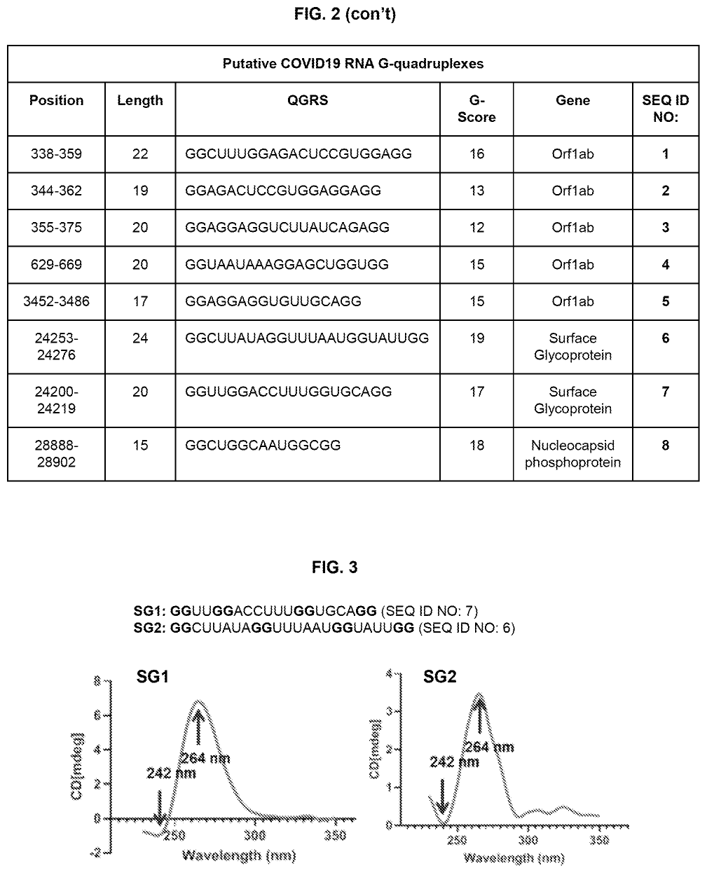

Analysis using Quadruplex forming G-Rich Sequences (QGRS) mapper identified the presence of at least eight highly conserved G-quadruplex forming regions (GQRs) in the Orf1ab, surface glycoprotein, and nucleocapsid phosphoprotein genes among different isolates of COVID-19 (FIG. 2). These genes encode the cell attachment glycoprotein, capsid, and RNA-dependent RNA polymerase, respectively, and are essential for viral entry and replication within host cells. G-quadruplexes and other secondary structures are highly important for many single-stranded RNA viral genomes, as these structures are known to interfere with protein translation. RNA-dependent RNA polymerase (RdRp) catalyzes the replication of positive-strand genomic COVID-19 RNA, as in other positive-strand RNA viruses. COVID-19 genome replication is a process of continuous synthesis that utilizes a full-length complementary negative-strand RNA as the template for progeny virus genomes.

Circular dichroism (CD) spectroscopy is a form of light absorption spectroscopy that measures the difference in absorbance of right- and left-circularly polarized light (rather than the commonly used absorbance of isotropic light) by a substance. Using CD spectroscopy, the formation of stable G-quadruplex structures from eight G-quadruplex forming regions was tested using two representative sequences, SG1 (24200-24219) and SG2 (24253-24276), in the presence of K⁺ (FIG. 3). The CD spectra of 5 μM SG1 and SG2 showed two well-defined signature peaks of right-handed parallel-stranded quadruplex RNA around 264 nm and 242 nm, which are characteristic of the stacking pattern and helicity, respectively.

Stabilization of RNA G4s by small molecules is anticipated to interfere with the synthesis of this complementary negative-strand RNA, with strong potential for diminishing the proliferation of the COVID-19 virus. Thus, the steric interaction of stabilization of GQRs by known G-quadruplex binding ligands represents a promising novel anti-COVID-19 strategy to inhibit the expression of GQR-harboring genes and thereby stop viral translation and replication.

### Example 1.2 Quinacrine as a New G4-Interactive Agent

Several G4-interactive agents have been identified from chemical libraries. Among them, quinacrine was selected (FIG. 4) since this drug has been approved for human use, implying that it is safe for human use for at least a couple of weeks. Quinacrine is also known to be lysosomotropic and can be enriched inside lysosomes, which are the primary organelles exploited by COVID-19 in viral uncoating and fusion. Lysosomotropic agents are generally weak bases that penetrate lysosomes in protonated form and increase intracellular pH. Thus, cationic G4-interactive agents, such as quinacrine, can inhibit COVID-19 viral entry at cellular lysosomes.

The G4-stabilizing effect of quinacrine was validated with the RET G4 as a model G4 using a DNA polymerase stop assay and CD spectroscopic studies. In this assay, the ligand mediated stabilization of the G-quadruplex structure arising from the DNA template prevents the progression of the Taq DNA polymerase during primer extension. As shown in FIG. 5, in the presence of quinacrine at increasing concentrations (0, 0.5, 1.0, and 2.0 μM), a dose-dependent increase in the amount of arrested product was observed, indicating the potential stabilization of the G4 structures by this compound.

Quinacrine was further investigated as to whether it stabilizes the RET G4 structure using circular dichroism (CD) spectroscopic analysis. As shown in FIG. 6, the positive peak at 262 nm, which corresponds to a parallel G-quadruplex structure, was not affected in the presence of quinacrine, suggesting that the parallel configuration of the RET G4 was not changed in the presence of this molecule. Next, the thermal stability of the G-quadruplex structure was examined by monitoring the CD melting curve in the absence and presence of quinacrine (5 equivalent) at increasing temperatures. As shown in FIG. 6, the melting temperature (Tm) of the RET G4 structure in the presence of quinacrine was significantly increased.

### Example 1.3 Spectrophotometry Study on the Interaction of Quinacrine with G4-RNA

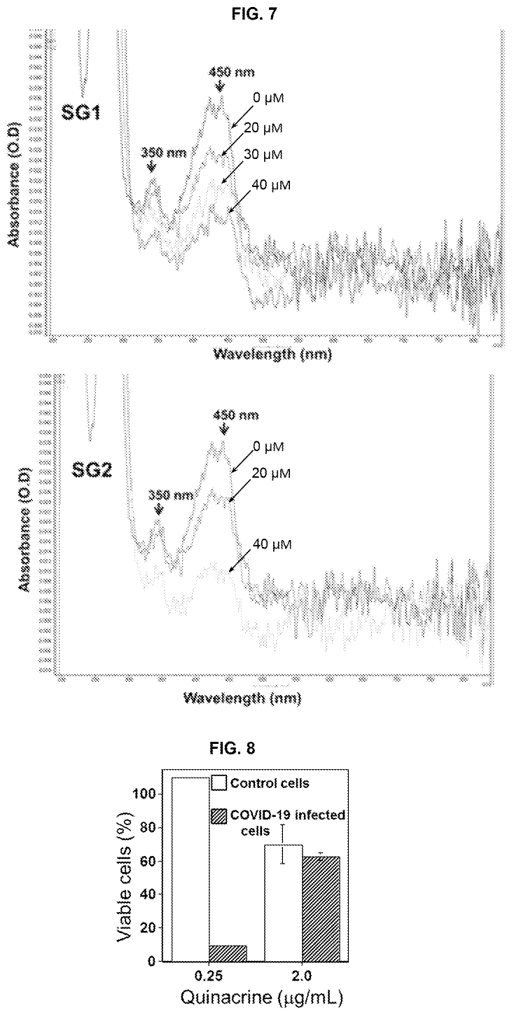

UV-Vis spectroscopy is the most common and convenient technique to study the interaction between small molecules with nucleic acids, including RNA G4. Molecules containing aromatic chromophore groups interact with RNA G4, and the molecular interaction can be studied based on the changes in the absorption spectra. The hypochromic effect is a spectral property for RNA G4-drug interactions that are closely related to the G4 structure. In order to prove the interaction between quinacrine and RNA G4, UV-Vis absorption spectra of quinacrine in the presence of different concentrations of two representative COVID-19 RNA G4s (SG1 and SG2) were recorded (FIG. 3). In the absence of RNA G4, quinacrine displayed two absorbance peaks at 350 and 450 nm, respectively (FIG. 7). With the addition of G4 RNA, the intensity at both 350 nm and 450 nm bands decreased, inducing the hypochromicity at these wavelengths (FIG. 7). This can be attributed to the decrease in the number of chromophores in solution due to the interaction of quinacrine with RNA G4.

### Example 1.4 Determination of the Antiviral Activities of the Test Drugs Against COVID-19 In Vitro

In this study, the antiviral efficiency of quinacrine was evaluated against COVID-19 in vitro. Antiviral activity was evaluated using a cytopathic effect (CPE) assay, which determines the ability of the compound to prevent viral CPE in African green monkey kidney cells, Vero E6. Four dilutions of the test compound were evaluated, and the effective antiviral concentration was determined by regression analysis. The cytotoxicity of the test compound was determined in parallel by the neutral red cell cytotoxicity assay, a common method used to detect cell viability or drug cytotoxicity. CPE was determined by the uptake of neutral red dye. Quinacrine protects more than 80% of the infected cells from the cytopathic effects caused by COVID-19 at 2 μg/mL concentration (FIG. 8). The tissue distribution of quinacrine was investigated in a previous study by treating mice daily by oral gavage with 30 mg/kg of quinacrine (35% v/v ethanol in distilled water) dissolved in 100 μL total volume for 28 days. After 28 d of treatment, the mean lung concentration for quinacrine was 2600 ng/mL (20), compared to 470 ng/ml in whole blood, implying that the mean concentration in the lung was significantly higher than those in whole blood. These data indicate that quinacrine can distribute into the lungs at concentrations that effectively suppress the proliferation of the COVID-19 virus.

### Example 1.5 Quinacrine in Combination with Remdesivir

Remdesivir displays potent in vitro activity against COVID-19 with an EC50 at 48 hours of 0.77 μM in MR-5 cells. The active triphosphate nucleoside form of remdesivir binds to RNA-dependent RNA polymerase and acts as an RNA-chain terminator, while quinacrine could alter the conformation of the template RNA for translation and replication. Without wishing to limit the present invention to a particular theory or mechanism, combining different therapeutic agents with different modes of action for viral disease can produce superior therapeutic rates with improved rates of overall survival. Thus, combining quinacrine with remdesivir may produce superior therapeutic rates with improved rates of overall survival.

### Example 2: Intravenous Administration of the Quinacrine

The following example describes treatment strategies for COVID-19 involving an intravenous administration of Quinacrine to a patient.

A 35-year-old man wakes up one morning with a fever of 102.4° F. and a tightness in his chest. He is an essential hospital worker and so believes he may have contracted COVID-19. Therefore, he calls his primary care physician to determine his next steps. The primary care physician decides that he should be brought in for testing since he is at a higher risk for complications because the patient has severe asthma. The man gets a nose swab for the test. The doctor mentions that she will call the patient with the results as soon as possible. For now, the man is told to self-isolate and to monitor any changes in symptoms, calling if things get progressively worse. Quickly the man's condition deteriorates, and he begins to experience difficulty breathing and pressure in his chest. The doctor admits him to the ICU of the hospital because he requires supplemental oxygen to maintain his oxygen levels. Shortly after being admitted to the ICU, the result of his test comes back positive for COVID-19. The ICU doctor gives the patient an intravenous injection of 200 mg initially every 6 hours for 5 doses, followed by 100 mg 3 times a day for 6 days. Alternatively, 100 mg is administered via IV infusion 3 times a day for 5 to 7 days. Within a few days of taking quinacrine, the patient is taken off the supplemental oxygen. The patient continues to improve, and after a week, the patient tests negative for COVID-19. The patient is released from the hospital and is told to stay in self-isolation for another week. No side effects are reported.

### Example 3—Oral Administration of Quinacrine

The following example describes treatment strategies for COVID-19 involving an oral administration of Quinacrine.

Recently, a COVID-19 test for a 36-year-old woman has come back positive. She has already been under surveillance at the hospital to make sure her condition does not get worse. After a week at the hospital, the patient's symptoms show no signs of improvement. The doctors at the hospital prescribe to the patient an initial 200 mg dose orally every 6 hours for 5 doses, followed by 100 mg 3 times a day for 6 days. Alternatively, 100 mg is orally administered to the patient after meals 3 times a day for 5 to 7 days. After a week and a half of taking quinacrine daily, the patient's symptoms improve. The patient continues to improve, and after a week, the patient tests negative for COVID-19. The patient is released from the hospital and is told to stay in self-isolation for another week. No side effects are reported.

### Example 4—RNA G4 Structures of Influenza a (H1N1) Genomic RNA

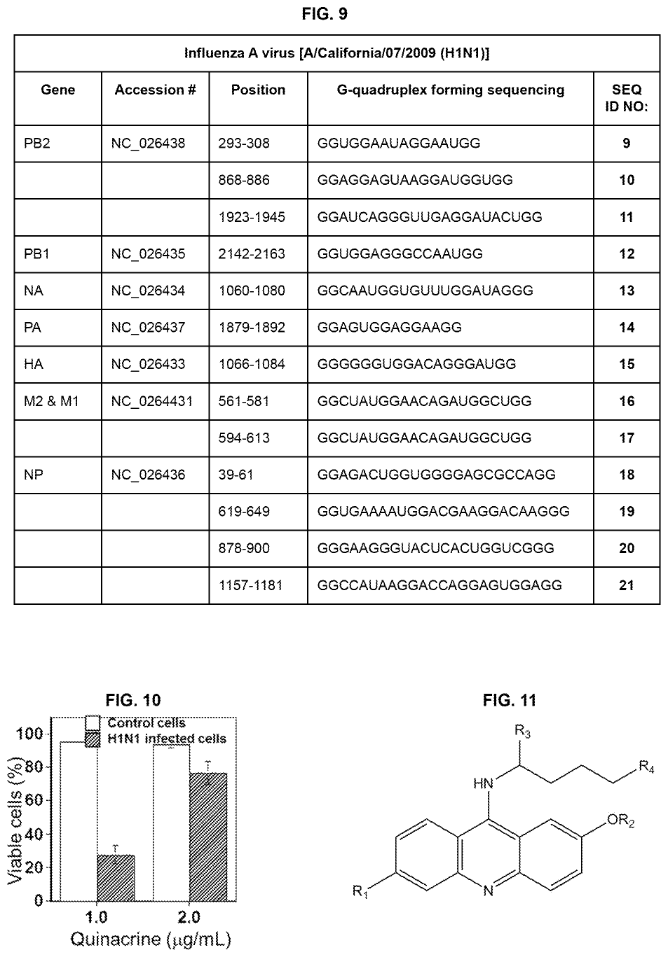

Since the introduction of the influenza A (H1N1) virus in 2009, H1N1 has circulated seasonally in the U.S., causing illnesses, hospitalizations, and deaths. Recent analysis using a QGRS mapper has identified the presence of at least thirteen highly conserved G-quadruplex forming regions (GQRs) in various genes of the H1N1 sub-strain (FIG. 9). These genes encode PB2, PB1, NA, PA, HA, M2 and MM1, and NP, respectively, and are essential for viral entry and replication within host cells (see accession number of each gene). Thus, the presence of these G-quadruplexes structures strongly suggests that quinacrine could interfere with protein translation and replication of positive-strand genomic influenza A RNA, as in other positive-strand RNA viruses and the COVID-19 virus.

### Example 4.1—Determination of the Antiviral Activities of the Test Drugs Against Influenza A (H1N1) In Vitro

In this study, the antiviral efficiency of quinacrine was evaluated against Influenza A (H1N1) in vitro. Antiviral activity was evaluated using a cytopathic effect (CPE) assay, which determines the ability of the compound to prevent viral CPE in African green monkey kidney cells, Vero E6. Four dilutions of test compounds were evaluated together with the cytotoxicity test by the neutral red cell cytotoxicity assay, a common method used to detect cell viability or drug cytotoxicity. Quinacrine protects more than 80% of the infected cells from the cytopathic effects caused by Influenza A (H1N1) at 2 μg/mL concentration (FIG. 10).

### Example 4.2—Potential Use of Quinacrine for the Treatment of SARS-COV-2 and Influenza Virus Co-Infection

COVID-19 mimics the many aspects of the influenza virus regarding clinical presentation, transmission mechanism, and seasonal coincidence. Thus, co-infection by both viruses is feasible. Currently, there is no therapy known for co-infection. Here, the present invention presents quinacrine as a potential therapy for the cases of SARS-COV-2 and influenza co-infection.

### Example 5—Oral Administration of Quinacrine

The following example describes prevention strategies for COVID-19 and influenza involving an oral administration of Quinacrine.

A 45 year-old man wants to prevent getting sick during the upcoming winter season. Therefore, he schedules an appointment with his primary care physician to discuss his options. At the appointment, the doctor recommends taking a 300 mg pill of quinacrine orally twice a day for the next month. The man successfully makes it through the winter season without getting sick. No side effects are reported

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-4. (canceled)

5. A method of preventing or treating a viral respiratory infection in a subject in need of such treatment, the method comprising administering to the subject a therapeutic amount of quinacrine or a derivative thereof.

6. The method of any one of claim 5, wherein the viral respiratory infection is caused by one or more types of viruses.

7. The method of claim 5, wherein the viral respiratory infection is caused by severe acute respiratory syndrome coronavirus 2 (SARS-COV2) virus, an influenza virus, respiratory syncytial virus, or a combination thereof.

8.-9. (canceled)

10. The method of claim 5, wherein quinacrine or derivative thereof is administered intravenously or orally.

11.-14. (canceled)

15. The method of claim 5, wherein the derivative of quinacrine is according to formula 1: 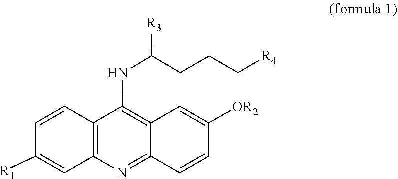
wherein R1 comprises H, F, or Cl group, wherein R2 comprises H or an alkyl group, wherein R3 comprises H or an alkyl group, and wherein R4 comprises an aliphatic amine or a nitrogen heterocyclic group; wherein the aliphatic amine or the nitrogen heterocyclic group comprises nitrogen, carbon, oxygen, and/or sulfur molecules.

16. (canceled)

17. The method of claim 5, wherein the derivative of quinacrine is according to any one of the following: 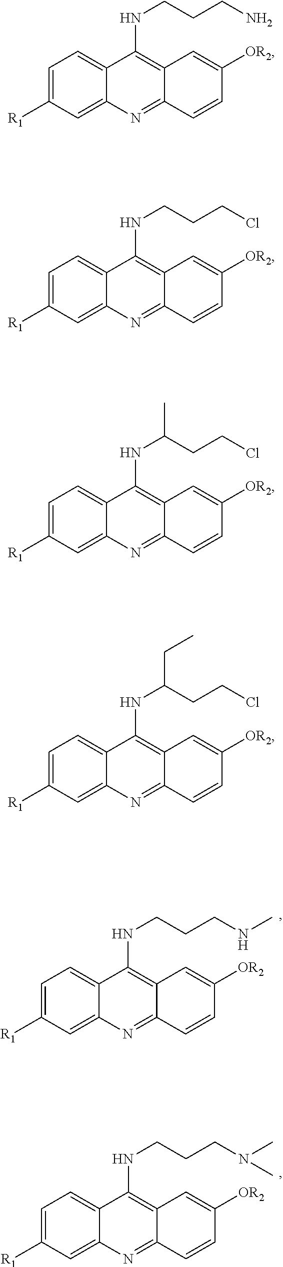 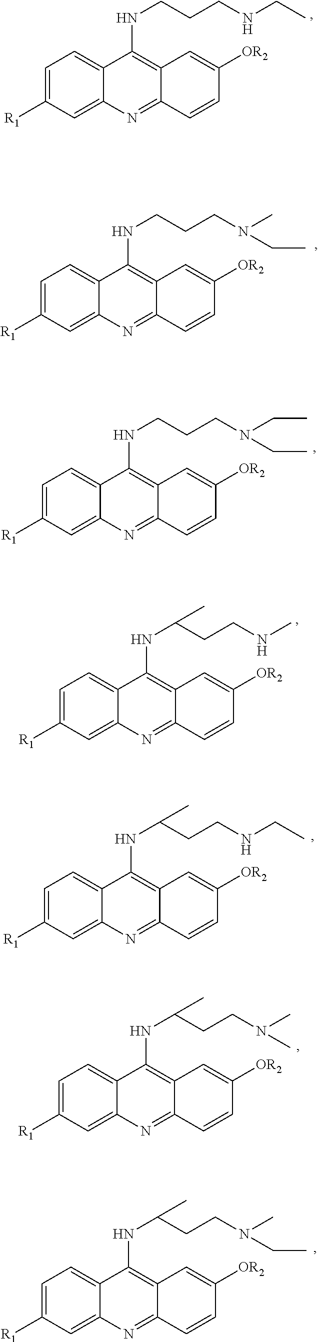 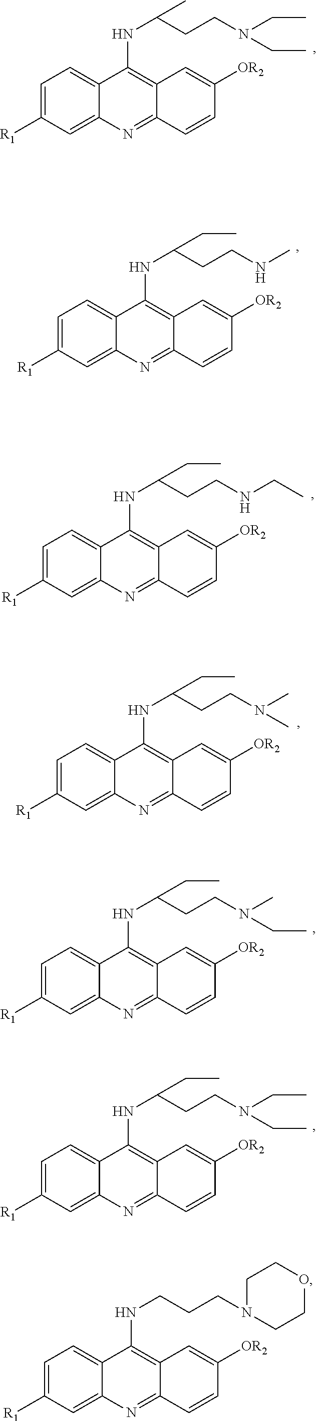 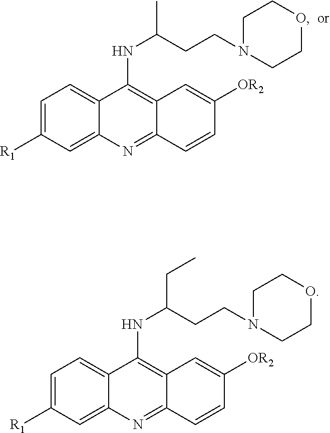
wherein R1 comprises H, F, or Cl group and wherein R2 comprises H or an alkyl group.

18.-21. (canceled)

22. The method of claim 5, wherein the derivative of quinacrine is according to any one of the following: 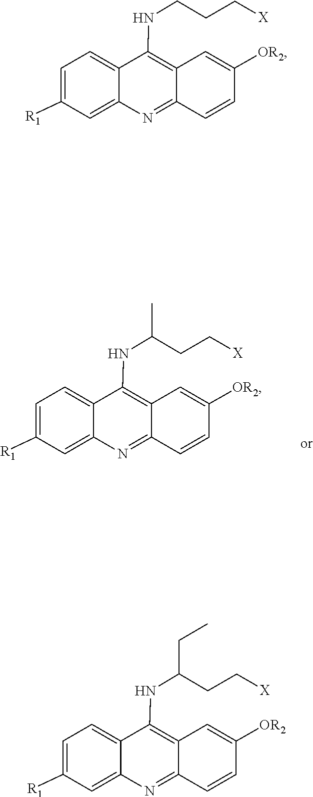
wherein R1 comprises H, F, or Cl group, wherein R2 comprises H or an alkyl group and X comprises a heterocyclic compound.

23. The method of claim 22, wherein the heterocyclic compound is according to any one of the following:

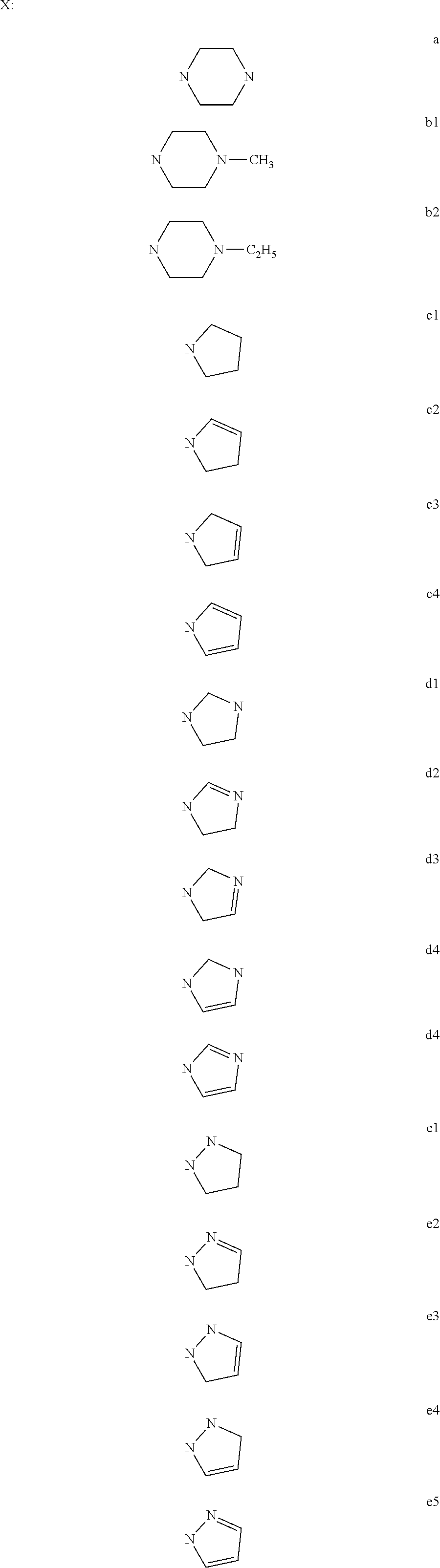

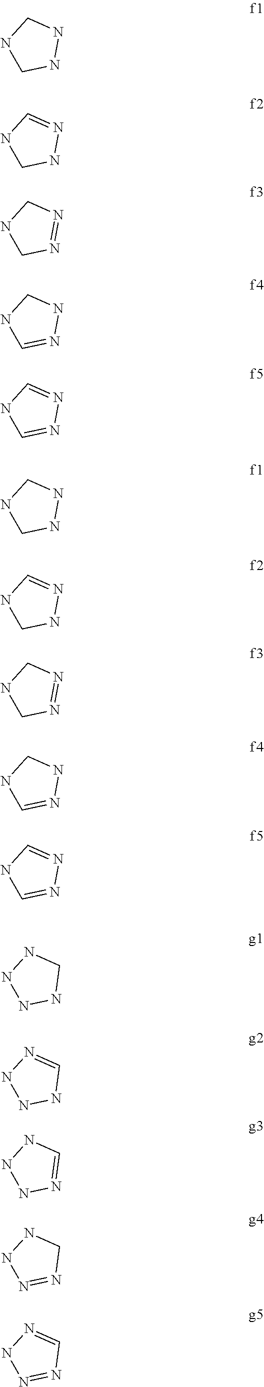

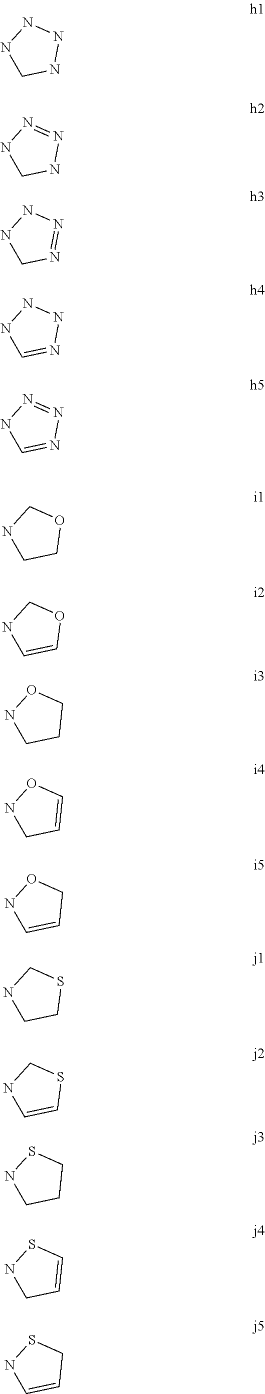

24. A composition comprising a derivative of quinacrine:

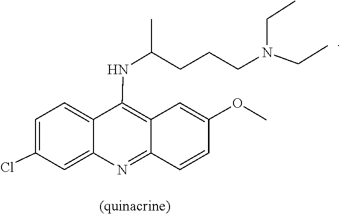 [SMILES: CCN(CC)CCCC(C)Nc1c2ccc(Cl)cc2nc2ccc(OC)cc12]

25. The composition of claim 24, wherein the derivative of quinacrine is according to formula 1: 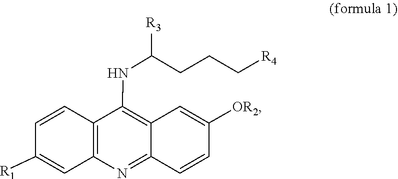
wherein R1 comprises H, F, or Cl group, wherein R2 comprises H or an alkyl group, wherein R3 comprises H or an alkyl group, and wherein R4 comprises an aliphatic amine or a nitrogen heterocyclic group.

26. The composition of claim 25, wherein the aliphatic amine or the nitrogen heterocyclic group comprises nitrogen, carbon, oxygen, and/or sulfur molecules.

27. The composition of claim 24, wherein the derivative of quinacrine is according to any one of the following: 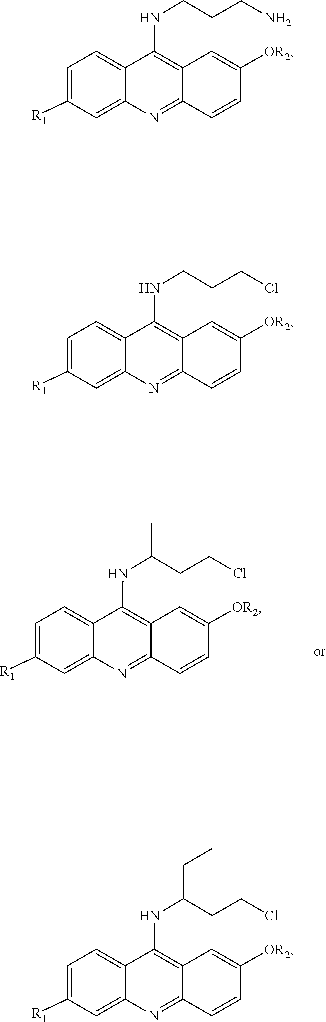
wherein R1 comprises H, F, or Cl group and wherein R2 comprises H or an alkyl group.

28. The composition of claim 24, wherein the derivative of quinacrine is according to any one of the following: 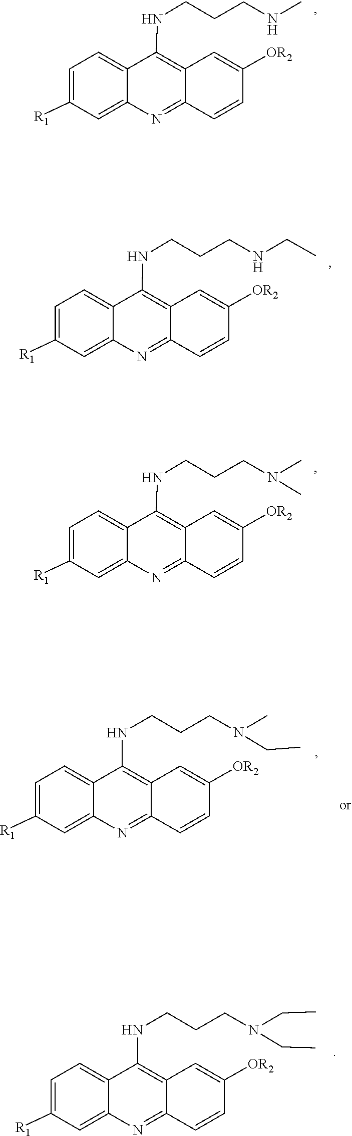
wherein R1 comprises H, F, or Cl group and wherein R2 comprises H or an alkyl group.

29. The composition of claim 24, wherein the derivative of quinacrine is according to any one of the following: 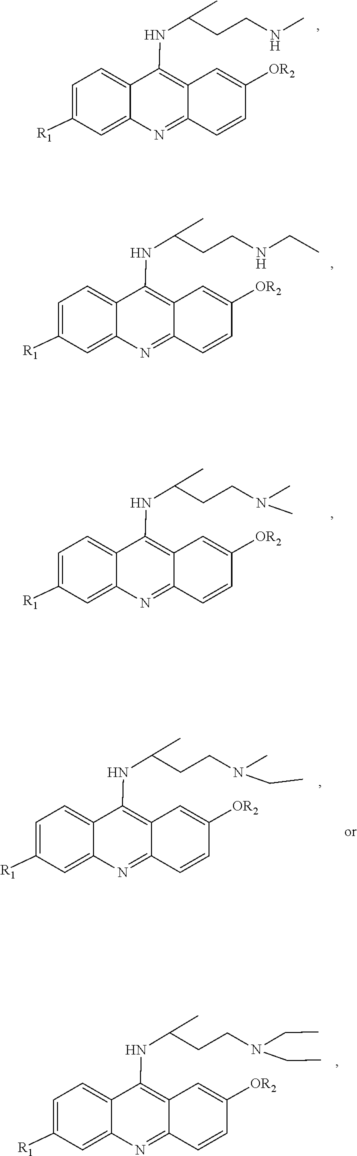
wherein R1 comprises H, F, or Cl group and wherein R2 comprises H or an alkyl group.

30. The composition of claim 24, wherein the derivative of quinacrine is according to any one of the following: 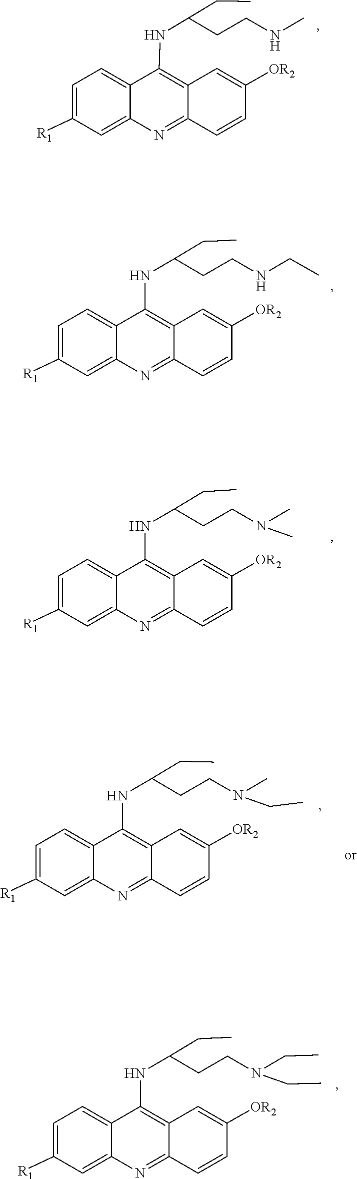
wherein R1 comprises H, F, or Cl group and wherein R2 comprises H or an alkyl group.

31. The composition of claim 24, wherein the derivative of quinacrine is according to any one of the following: 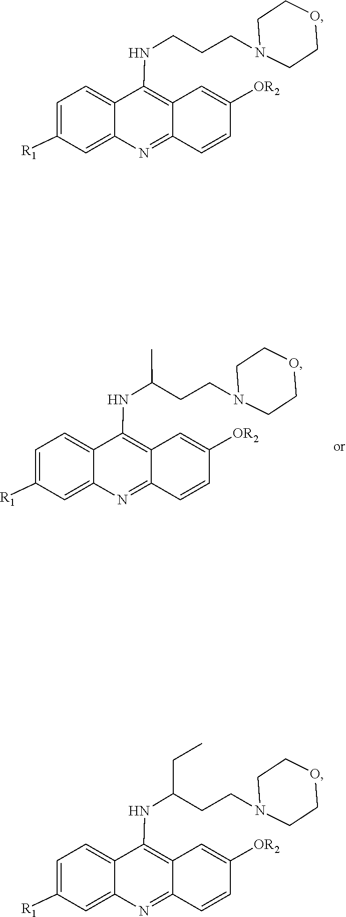
wherein R1 comprises H, F, or Cl group and wherein R2 comprises H or an alkyl group.

32. The composition of claim 24, wherein the derivative of quinacrine is according to any one of the following: 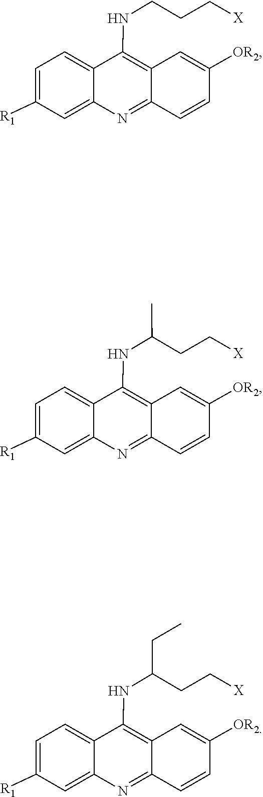
wherein R1 comprises H, F, or Cl group, wherein R2 comprises H or an alkyl group and X comprises a heterocyclic compound.

33. The composition of claim 32, wherein the heterocyclic compound is according to any one of the following:

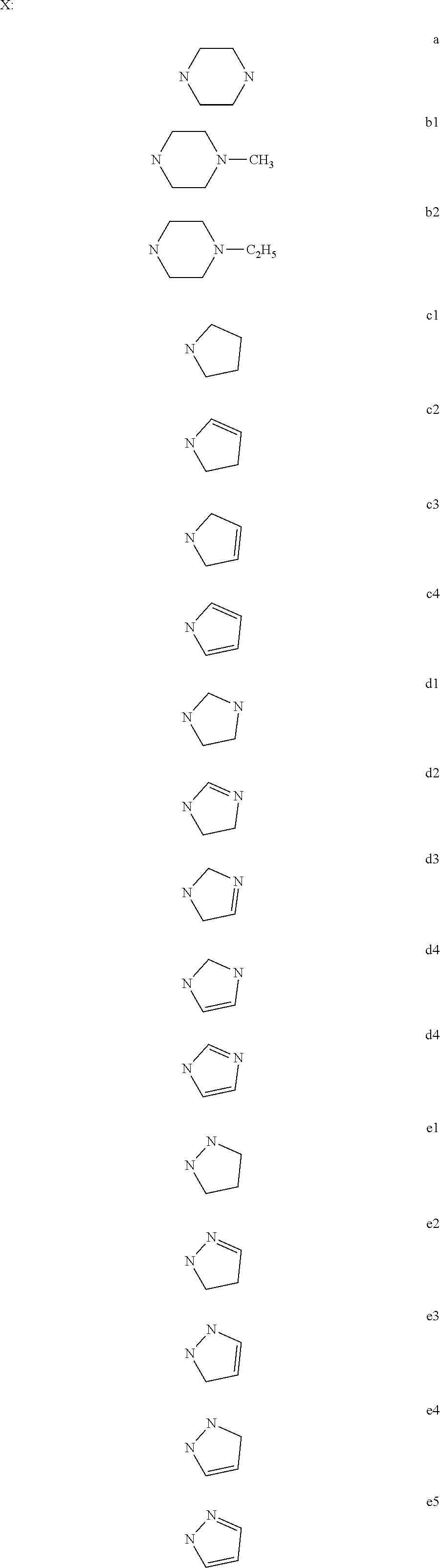

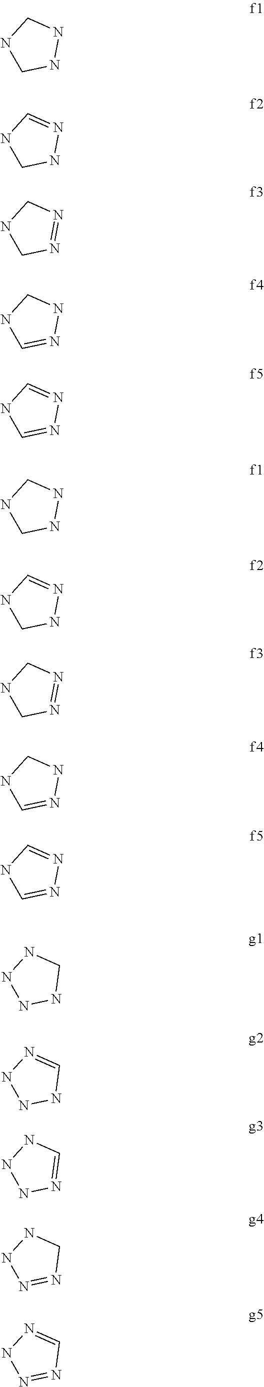

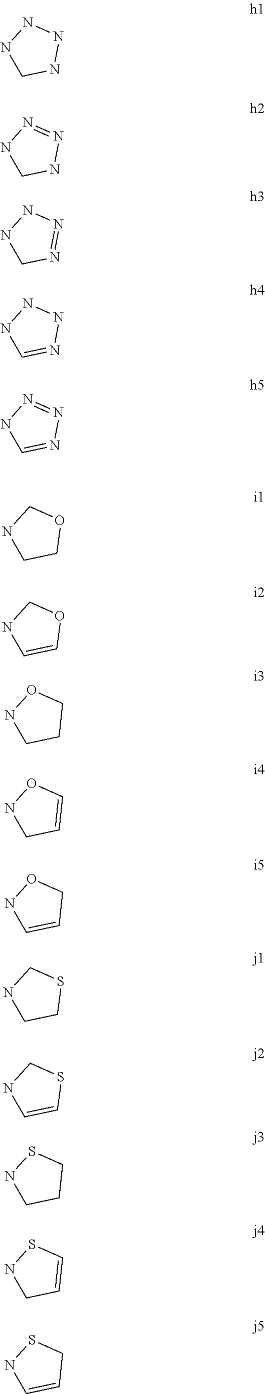

34. A method of preventing or treating coronavirus disease 2019 (COVID-19) in a subject in need of such treatment, the method comprising administering to the subject a therapeutic amount of a composition according to claim 24.

35. A method of simultaneously treating both coronavirus disease 2019 (COVID-19) and influenza in a subject in need of such treatment, the method comprising administering to the subject a therapeutic amount of a composition according to claim 24.

36.-50. (canceled)


SEQUENCE LISTING (21 sequences)

SEQ ID NO: 1 - RNA, 22 nt, Coronavirus HKU15
  source 1..22: mol_type = mRNA
ggctttggagactccgtggagg

SEQ ID NO: 2 - RNA, 19 nt, Coronavirus HKU15
  source 1..19: mol_type = mRNA
ggagactccgtggaggagg

SEQ ID NO: 3 - RNA, 20 nt, Coronavirus HKU15
  source 1..20: mol_type = mRNA
ggaggaggtcttatcagagg

SEQ ID NO: 4 - RNA, 20 nt, Coronavirus HKU15
  source 1..20: mol_type = mRNA
ggtaataaaggagctggtgg

SEQ ID NO: 5 - RNA, 17 nt, Coronavirus HKU15
  source 1..17: mol_type = mRNA
ggaggaggtgttgcagg

SEQ ID NO: 6 - RNA, 24 nt, Coronavirus HKU15
  source 1..24: mol_type = mRNA
ggcttataggtttaatggtattgg

SEQ ID NO: 7 - RNA, 20 nt, Coronavirus HKU15
  source 1..20: mol_type = mRNA
ggttggacctttggtgcagg

SEQ ID NO: 8 - RNA, 15 nt, Coronavirus HKU15
  source 1..15: mol_type = mRNA
ggctggcaatggcgg

SEQ ID NO: 9 - RNA, 16 nt, unidentified
  source 1..16: mol_type = mRNA; strain = A/California/07/2009 (H1N1)
ggtggaataggaatgg

SEQ ID NO: 10 - RNA, 19 nt, unidentified
  source 1..19: mol_type = mRNA; strain = A/California/07/2009 (H1N1)
ggaggagtaaggatggtgg

SEQ ID NO: 11 - RNA, 22 nt, unidentified
  source 1..22: mol_type = mRNA; strain = A/California/07/2009 (H1N1)
ggatcagggttgaggatactgg

SEQ ID NO: 12 - RNA, 16 nt, unidentified
  source 1..16: mol_type = mRNA; strain = A/California/07/2009 (H1N1)
ggtggagggccaatgg

SEQ ID NO: 13 - RNA, 21 nt, unidentified
  source 1..21: mol_type = mRNA; strain = A/California/07/2009 (H1N1)
ggcaatggtgtttggataggg

SEQ ID NO: 14 - RNA, 14 nt, unidentified
  source 1..14: mol_type = mRNA; strain = A/California/07/2009 (H1N1)
ggagtggaggaagg

SEQ ID NO: 15 - RNA, 19 nt, unidentified
  source 1..19: mol_type = mRNA; strain = A/California/07/2009 (H1N1)
ggggggtggacagggatgg

SEQ ID NO: 16 - RNA, 21 nt, unidentified
  source 1..21: mol_type = mRNA; strain = A/California/07/2009 (H1N1)
ggctatggaacagatggctgg

SEQ ID NO: 17 - RNA, 21 nt, unidentified
  source 1..21: mol_type = mRNA; strain = A/California/07/2009 (H1N1)
ggctatggaacagatggctgg

SEQ ID NO: 18 - RNA, 23 nt, unidentified
  source 1..23: mol_type = mRNA; strain = A/California/07/2009 (H1N1)
ggagactggtggggagcgccagg

SEQ ID NO: 19 - RNA, 25 nt, unidentified
  source 1..25: mol_type = mRNA; strain = A/California/07/2009 (H1N1)
ggtgaaaatggacgaaggacaaggg

SEQ ID NO: 20 - RNA, 23 nt, unidentified
  source 1..23: mol_type = mRNA; strain = A/California/07/2009 (H1N1)
gggaagggtactcactggtcggg

SEQ ID NO: 21 - RNA, 24 nt, unidentified
  source 1..24: mol_type = mRNA; strain = A/California/07/2009 (H1N1)
ggccataaggaccaggagtggagg